(19) United States
(12) Patent Application Publication
(10) Pub. No.: US 2026/0219339 A1
(43) Pub. Date: Jul. 30, 2026

(54) MAGNETIC PARTICLE SPECTROSCOPY METHOD AND DEVICE

(71) Applicant: Regents of the University of Minnesota, Minneapolis, MN (US)

(72) Inventors: Jian-Ping Wang, Shoreview, MN (US); Vinit Kumar Chugh, Minneapolis, MN (US); Kai Wu, Saint Paul, MN (US)

(21) Appl. No.: 19/145,163

(22) PCT Filed: Jan. 5, 2024
(86) PCT No.: PCT/US2024/010484

§ 371 Date: Jul. 1, 2025

Related U.S. Application Data
(60) Provisional application No. 63/478,860, filed on Jan. 6, 2023.

(51) Int. Cl. G01R 33/12; G01N 27/74; G01N 33/543

(52) U.S. Cl. CPC G01R 33/1269; G01N 27/745; G01N 33/54326

(57) ABSTRACT

A method includes separating a bound magnetic nanoparticle (MNP) from an unbound MNP. The bound MNP includes an MNP bound to an analyte, and a surface functionalization including a probe configured to bind to the analyte.

This application claims the benefit of U.S. Provisional Patent Application 63/478,860, filed 6 Jan. 2023, the entire content of which is incorporated herein by reference.

### GOVERNMENT RIGHTS

This invention was made with government support under DE030832 awarded by the National Institutes of Health, and under 2020-67021-31956 awarded by the National Institute of Food and Agriculture. The government has certain rights in the invention.

### TECHNICAL FIELD

This disclosure relates to magnetic particle spectroscopy.

### BACKGROUND

Bioassays are procedures for detecting or measuring the concentration or potency of a substance by its effect on living cells or tissues. Immunoassays are procedures for detecting or measuring specific proteins or other substances through their properties as antigens and/or antibodies. In some instances, immunoassays, among other tests, may be performed using magnetic particle spectroscopy (MPS).

### SUMMARY

In general, this disclosure describes example magnetic particle spectroscopy (MPS) devices and techniques for detecting chemicals and biological substances via MPS. MPS-based volumetric assays may use surface functionalized magnetic nanoparticles (MNPs), functionalized with probes, mixed with a fluidic sample that contains target analytes of interest. The specially designed probes (e.g., antibody, antigen, peptide, DNA, RNA, or the like) of the functionalized MNPs surfaces may specifically bind to target analytes due to antibody-protein, or DNA-DNA, or DNA-protein interactions and form bound MNPs and/or clusters/conjugates. Although a one-step, wash-free volumetric assay may be easy to handle by a layperson, the detection sensitivity may be impaired by the remaining unbound MNPs. For example, the magnetic signal of bound MNPs changes, e.g., Brownian relaxation of a bound MNP may be reduced and/or blocked due to cross-linking in a cluster, while the signal from unbound MNPs is unchanged. The unbound MNPs may generate higher magnetic signals that may cause limitation to signal detection, such as analog-to-digital (ADC) resolution. The relatively small changes due to bound MNPs may then be unresolvable or unidentifiable leading to sensitivity limitations. Furthermore, the detection methodology for the assay may be governed by Brownian relaxation of MNPs.

Also, MPS-based volumetric assay may rely on the probe-analyte binding to block the Brownian relaxation of MNPs, thus, the magnetic signal change can be quantitatively correlated to the amount/concentration of target analytes in the fluid. This design may limit choices of MNPs that may be used that show Brownian relaxation, and the MNPs may have a size on the order of several tens of nanometers.

In examples described herein, bound MNPs and/or MNP conjugates/clusters may be separated and/or isolated from unbound MNPs, e.g., in a post-assay treatment and/or sample preparation step. After removing unbound MNPs, Néel relaxation phenomena of MNPs in conjugates may be used, thus providing a larger magnetic signature per MNP. The bound MNPs and/or isolated MNP conjugates/clusters may be enriched or even dried, and Brownian relaxation may no longer required, increasing options of MNPs that may be used in an assay. For example, multicore MNPs with larger overall sizes (up to several micrometers) and much higher magnetic moments per particle may be used with the techniques described herein.

The magnetic signal from these MNPs may be caused by Néel relaxation, e.g., rather than Brownian relaxation. The signal level may proportional to the amount of MNPs conjugated/bound to target analytes. Higher assay sensitivity may be achieved by using larger MNPs that show higher magnetic moment per particle. In addition, different MNPs (e.g., based on material, size, shape, single- or multi-core structure, or the like) with unique MPS spectra may be used, e.g., allowing multiplexed detection by virtue of magnetic colorization. In addition to improved detectivity, separation of bound MNPs from unbound MNPs may allow for analyzing liquid samples of larger volumes bound MNPs. For example, MPS devices may be limited in the maximum volume of sample that can be measured by conductive coil geometry, and separating bound MNPs from unbound MNPs to increase the concentration of bound MNPs may effectively increase the volume of biological sample. Meanwhile, the surface functionalized MNPs that may be measured by replacing a volume of fluid including a relatively lower concentration of bound MNPs and a relatively higher concentration of unbound MNPs with a volume of fluid having a relatively higher concentration of bound MNPs and a relatively lower concentration of unbound MNPs.

In some examples, the disclosure describes a bioassay system including: at least one conductive excitation coil, the at least one conductive excitation coil configured to generate an alternating magnetic field including a first frequency and a second frequency; a sample mount configured to position a sample within the at least one conductive excitation coil; a separator configured to separate a bound magnetic nanoparticle (MNP) from an unbound MNP, wherein the bound MNP comprises a surface functionalized MNP bound to an analyte, wherein the unbound MNP comprises a surface functionalized MNP not bound to an analyte, wherein the sample comprises the bound MNP and does not comprise the unbound MNP; and at least one sensing conductive coil configured to sense a magnetic response of the sample positioned within the sample mount to the alternating magnetic field.

In some examples, the disclosure describes a method including: positioning a sample within at least one conductive excitation coil, wherein the at least one conductive excitation coil is configured to generate an alternating magnetic field including a first frequency and a second frequency; separating a bound magnetic nanoparticle (MNP) from an unbound MNP, wherein the bound MNP comprises a surface functionalized MNP bound to an analyte, wherein the unbound MNP comprises a surface functionalized MNP not bound to an analyte, wherein the sample comprises the bound MNP and does not comprise the unbound MNP; and sensing, by at least one sensing conductive coil, a magnetic response of the sample to the alternating magnetic field.

In some examples, the disclosure describes a bioassay system including: at least one conductive excitation coil, the at least one conductive excitation coil configured to generate an alternating magnetic field including a first frequency and a second frequency; a sample mount configured to position a sample within the at least one conductive excitation coil, wherein the sample comprises a bound magnetic nanoparticle (MNP) that has been separated from an unbound MNP, wherein the sample does not comprise the unbound MNP, wherein the bound MNP comprises a surface functionalized MNP bound to an analyte, wherein the unbound MNP comprises a surface functionalized MNP not bound to an analyte; at least one sensing conductive coil configured to sense a magnetic response of the sample positioned within the sample mount to the alternating magnetic field.

In some examples, the disclosure describes a method including: separating a bound magnetic nanoparticle (MNP) from an unbound MNP, wherein the bound MNP comprises an MNP bound to an analyte, wherein the bound MNP comprises a surface functionalization including a probe configured to bind to the analyte.

In some examples, the disclosure describes a separation apparatus including: a container configured to house a sample comprising a bound magnetic nanoparticle (MNP) and an unbound MNP, wherein the bound MNP comprises an MNP bound to an analyte, wherein the bound MNP comprises a surface functionalization including a probe configured to bind to the analyte, wherein the bound MNP has a hydrodynamic size that is larger than the unbound MNP; and a separator configured to separate, based on hydrodynamic size, the bound MNP from the unbound MNP.

In some examples, the disclosure describes a magnetic nanoparticle (MNP) test material comprising: a surface functionalized MNP comprising a surface functionalization including a probe configured to bind to an analyte; and a nonmagnetic microstructure configured to bind to the analyte.

Thus, the disclosed embodiments provide MPS techniques for one step, wash-free immunoassays with improved portability and sensitivity and reduced cost and complexity.

### BRIEF DESCRIPTION OF THE DRAWINGS

FIG. 1 is an illustration of an example diagnosis system including an MPS handheld device, in accordance with one or more techniques of this disclosure.

FIG. 2 is a block diagram of example circuit of an example MPS handheld device, in accordance with one or more techniques of this disclosure.

FIG. 3 is a block diagram of another example circuitry of an example MPS handheld device, in accordance with one or more techniques of this disclosure.

FIG. 4A is a schematic cross-sectional illustration of a portion of an example MPS handheld device, in accordance with one or more techniques of this disclosure.

FIG. 4B is an illustration of an example plot of the amplitude of a composite excitation magnetic field as a function of frequency, in accordance with one or more techniques of this disclosure.

FIG. 4C is an illustration of an example plot of the amplitude of magnetic responses from MNPs detected by pick-up coils as a function of frequency, in accordance with one or more techniques of this disclosure.

FIG. 5A is an illustration an example plot of the amplitude of two alternating magnetic fields generated by drive coils as a function of time, in accordance with one or more techniques of this disclosure.

FIG. 5B is an illustration an example plot of the amplitude of composite magnetic field as a function of frequency, in accordance with one or more techniques of this disclosure.

FIG. 5C is an illustration of an example plot of the magnetic response of MNPs as a function of magnetic field, in accordance with one or more techniques of this disclosure.

FIG. 5D is an illustration an example plot of the amplitude of a magnetic response of one or more MNPs within the generated magnetic field of FIG. 5A as a function of time, in accordance with one or more techniques of this disclosure.

FIG. 5E is an illustration of an example plot of the amplitude of the magnetic response of FIG. 5D as a function of frequency, in accordance with one or more techniques of this disclosure.

FIG. 6A is an illustration of an example plot of the harmonic amplitude response as a function of hydrodynamic size of MNPs, in accordance with one or more techniques of this disclosure.

FIG. 6B is an illustration of an example Neel motion of an MNP, in accordance with one or more techniques of this disclosure.

FIG. 6C is an illustration of an example Brownian motion of an MNP, in accordance with one or more techniques of this disclosure.

FIG. 7A is an illustration of an example bound MNP, in accordance with one or more techniques of this disclosure.

FIG. 7B is an illustration of another example bound MNP, in accordance with one or more techniques of this disclosure.

FIG. 7C is an illustration of another example bound MNP, in accordance with one or more techniques of this disclosure.

FIG. 7D is an illustration of another example bound MNP, in accordance with one or more techniques of this disclosure.

FIG. 7E is an illustration of another example bound MNP, in accordance with one or more techniques of this disclosure.

FIG. 8 is a flowchart of an example method of detecting chemicals and/or biological substances via MPS including separating bound MNPs from unbound MNPs, in accordance with one or more techniques of this disclosure.

FIG. 9 is a schematic illustration of steps of the method of FIG. 8, in accordance with one or more techniques of this disclosure.

FIG. 10 is a schematic diagram illustrating an example separator including a filter, in accordance with one or more techniques of this disclosure.

FIG. 11 is a schematic diagram illustrating an example magnetic separator, in accordance with one or more techniques of this disclosure.

FIG. 12 is a schematic diagram illustrating another example gravitational separator, in accordance with one or more techniques of this disclosure.

FIG. 13A is a schematic diagram illustrating another example centrifuge separator, in accordance with one or more techniques of this disclosure.

FIG. 13B is a schematic diagram illustrating an example container including separated bound MNPs and unbound MNPs after having been separated by the centrifuge separator of FIG. 13A, in accordance with one or more techniques of this disclosure.

FIG. 14 is a schematic diagram illustrating an example surface acoustic wave separator, in accordance with one or more techniques of this disclosure.

FIG. 15 is an illustration of an example surface functionalized MNP, in accordance with one or more techniques of this disclosure.

FIG. 16 is an illustration of example surface functionalized MNPs, in accordance with one or more techniques of this disclosure.

FIG. 17 is an illustration of an example surface functionalized MNP, in accordance with one or more techniques of this disclosure.

FIG. 18 is an illustration of an example surface functionalized MNP, in accordance with one or more techniques of this disclosure.

FIG. 19 is an illustration of an example surface functionalized MNP, in accordance with one or more techniques of this disclosure.

FIG. 20 is an illustration of example surface functionalized MNPs, in accordance with one or more techniques of this disclosure.

FIG. 21 is an illustration of an example surface functionalized MNP, in accordance with one or more techniques of this disclosure.

FIG. 22 is an illustration of example surface functionalized MNPs, in accordance with one or more techniques of this disclosure.

FIG. 23 is an illustration of an example surface functionalized MNPs, in accordance with one or more techniques of this disclosure.

FIG. 24 is an illustration of example surface functionalized microbead and MNP, in accordance with one or more techniques of this disclosure.

FIG. 25A is an illustration of example surface functionalized microbead and MNP, in accordance with one or more techniques of this disclosure.

FIG. 25B is an illustration of an example surface functionalized MNP, in accordance with one or more techniques of this disclosure.

FIG. 25C is an illustration of an example surface functionalized MNP, in accordance with one or more techniques of this disclosure.

FIG. 26 is an illustration of an exploded-view of an example MPS device, in accordance with one or more techniques of this disclosure.

FIG. 27 is an illustration of an example MPS device, in accordance with one or more techniques of this disclosure.

Like symbols in the drawings indicate like elements.

### DETAILED DESCRIPTION

In recent years, magnetic particle spectroscopy (MPS) has emerged as a new technology for immunoassay applications. In MPS, alternating magnetic fields may be applied to magnetic nanoparticles (MNPs). The magnetic responses of these nanoparticles may be collected and recorded by a pair of specially designed pick-up coils. These magnetic responses may contain higher harmonics that are specific to the physical changes of the nanoparticles, such as binding events of target analytes to nanoparticles. MPS may be a volumetric-based bioassay method that analyzes the response signal from the whole nanoparticle suspension. In examples, a handheld MPS system that may have high, or increased, sensitivity, reduced cost, may be performed in vitro, and may be an easy-to-use point-of-care (POC) detection kit is disclosed. Examples of handheld MPS systems are described in WO 2021/212144 entitled “MAGNETIC PARTICLE SPECTROSCOPY METHOD AND DEVICE,” the contents of which are incorporated by reference herein.

Magnetic particle imaging (MPI) has emerged as a new imaging modality that directly detects MNP tracers using alternating magnetic fields. MPS, a derivative technology of MPI, has emerged as a novel immunoassay tool that may be a wash-free, easy-to-use, and portable home healthcare modality. MPS may be interpreted as OD MPI, e.g., a spatially independent MPI, where a bi-directional sinusoidal magnetic field may be applied to a suspension of MNPs. The magnetization of MNPs relax in response to the external magnetic field through Brownian and Néel relaxations. These MNPs may be specially designed and coated with antibodies that specifically recognize and bind to target analytes from biofluid samples. Due to the nonlinear magnetic responses of these MNPs, higher harmonics may be picked up by a pair of specially designed pick-up coils. These harmonics may be extracted by means of appropriate filtering and may be indicators of the physical environments of MNPs, for example, the viscosity and temperature of the liquid medium as well as the binding events of target analytes onto MNPs. Since biological tissues and fluids are nonmagnetic, there may be negligible magnetic background noise from biological samples. MNPs may be the sole sources of magnetic responses from testing samples and this volumetric-based immunoassay tool may allow for reduced sample preparation.

In an MPS-based volumetric assay apparatus, surface functionalized magnetic nanoparticles (MNPs) are mixed with fluidic sample that contains target analytes of interest. Specially designed probes (e.g., antibody, antigen, peptide, DNA, RNA, etc.) on the surface-functionalized MNPs surfaces may specifically bind to target analytes due to antibody-protein, or DNA-DNA, or DNA-protein interactions and form clusters, e.g., clusters of MNPs with bound target analytes. A one-step, wash-free volumetric assay may be relatively easy to handle by a layperson. However, detection sensitivity of a one-step, wash-free volumetric assay may be impaired by unbound MNPs that may remain, e.g., within the volume along with the bound MNPs and bound MNP clusters. In examples described herein, MPS-based volumetric assay devices, systems, and techniques include a post-assay sample treatment to separate and isolate bound MNPs and/or bound MNP clusters (e.g., MNP conjugates) from unbound MNPs.

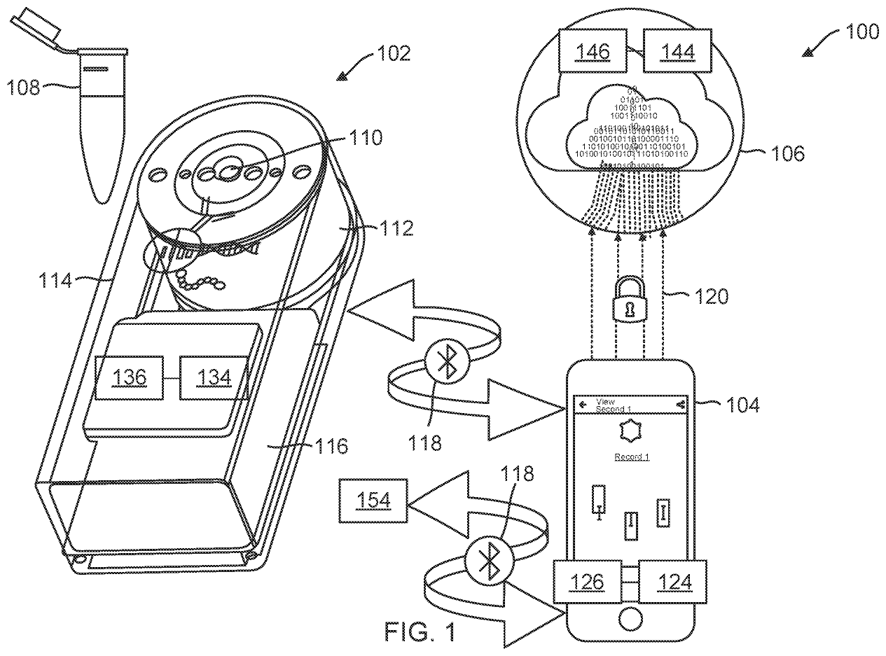

FIG. 1 is an illustration of an example diagnosis system 100 including an MPS handheld device 102, in accordance with one or more techniques of this disclosure. In the example shown, diagnosis system 100 includes MPS handheld device 102, a computing device 104, a distributed computing system 106, e.g., cloud computing system 106, a sample vial 108, and a separator 154.

In the example shown, MPS handheld device 102 includes a sample loading port 110, coils 112, a housing 114, and circuitry 116. Sample loading port 110 may be configured to accept samples, for example, sample vial 108, and to position the sample to be tested in the correct position with respect to coils 112 for an MPS measurement or measurements. Coils 112 may include drive coils, e.g., primary coils, secondary coils, etc., and pick-up coils, or any type of coil suitable for forming, controlling, and detecting or sensing magnetic fields. Coils 112 may be made of any suitable conductive and/or magnetic material, for example, copper, silver, aluminum, or the like. Housing 114 may be configured to enclose, support, and position the components of MPS handheld device 102 correctly with respect to each other, e.g., sample loading port 110 and coils 112, to provide a structure for connecting circuitry 116 to coils 112 and any other components of MPS handheld device 102, and to provide structure for a user physically manipulate MPS handheld device 102. In some examples, housing 114 may be 3D printed with any suitable material, such as a polymer. In some examples, the polymer may include polylactic acid (PLA). In some examples, MPS handheld device 102 may be manipulatable by hand by a user, e.g., MPS handheld device 102 may be a handheld device.

In some examples, sample vial 108 may include or contain a liquid including one or more MNPs. In some examples, material to be tested, e.g., a fluid such as a sample of a patient's blood or blood components, may be added to sample vial 108. The MNPs included in sample vial 108 may be surface functionalized via coating with ligands (e.g., carboxylic acid and amine, and the like), proteins (e.g., antibodies, polyclonal antibodies, streptavidin, protein A, and the like), antigens, nucleic acids (e.g., deoxyribonucleic acid (DNA), ribonucleic acid (RNA), or the like) or any combination thereof, or may be carried in a liquid. Sample vial 108 may be configured to have a long shelf life, for example, a shelf life greater than one hour, greater than one day, greater than one week, greater than one month, greater than one year, greater than ten years, or any other long shelf life. In some examples, the MNPs included in sample vial 108 may be configured to be stored at room temperature, or at lower temperatures, for example, near 4° Celsius (C). The MNPs included in sample vial 108 may be configured to be surface functionalized with different capture probes, e.g., antibodies, antigens, DNA, RNA, and the like, designed to detect one or more specific biomarkers, e.g., one or more specific disease. In some examples, sample vial 108 may be a flat bottom, USP type I glass vial, may have dimensions of 31 millimeters (mm) by 5 mm and a volume capacity of 0.25 milliliters (mL), and may be one-time use only, e.g., disposable. In other examples, sample vial 108 may be a plastic vial, or made of any other suitable material.

In some examples, MPS handheld device 102 may be communicatively coupled, for example by a wired or a wireless connection 118 and/or 120, to computing device 104 and/or distributed system 106. In some examples, connection 118 and/or 120 may be a secured connection, e.g., encrypted, requiring two-factor authentication, and the like. Measurements and/or information corresponding to measurements may be transferred to computing device 106 and/or distributed system 106, for example, for processing of measurements and/or information corresponding to measurements. In some examples, circuitry 116 of MPS handheld device 102 may include processing circuitry 136 and memory 134, and may process measurements and/or information corresponding to measurements without transferring the measurements and/or information corresponding to measurements to computing device 104 or distributed computing system 106. In some examples, computing device 104 may be communicatively coupled to distributed computing system 106, for example by a wired or a wireless connection 120, and measurements and/or information corresponding to measurements from MPS handheld device 102 received by computing device 104 may be transferred to distributed computing system 106, for example, for processing of measurements and/or information corresponding to measurements.

In the illustrated example, computing device 106 may include processing circuitry 126 coupled to memory 124 and to a display, one or more outputs, and one or more user inputs of a user interface. Processing circuitry 126, as well as processing circuitry 136, and other processing modules or circuitry described herein, e.g., processing circuitry 146 of distributed computing system 106, may be any suitable software, firmware, hardware, or combination thereof. Processing circuitry 126, 136, 146 may include any one or more microprocessors, controllers, digital signal processors (DSPs), application specific integrated circuits (ASICs), field-programmable gate arrays (FPGAs), or discrete logic circuitry. The functions attributed to processors described herein, including processing circuitry 126, may be provided by processing circuitry of a hardware device, e.g., as supported by software and/or firmware.

In some examples, processing circuitry 126, as well as processing circuitry 136, 146, may be configured to determine diagnosis information associated with MPS measurements and/or MPS measurement information. For example, the processing circuitry 126 may determine amplitudes and/or phases of magnetic fields detected via coils 112 and may perform any suitable signal processing to determine a diagnosis based on the amplitudes and/or phases of the magnetic fields. Processing circuitry 126 may also receive input signals from additional sources (not shown). For example, processing circuitry 126 may receive an input signal containing position information, such as Global Navigation Satellite System (GNSS) coordinates of MPS handheld device 102 and/or computing device 104. Additional input signals may be used by processing circuitry 126 in any of the calculations or operations it performs. In some examples, processing circuitry 126 may be adapted to execute software, which may include an operating system and one or more applications, as part of performing the functions described herein. In some examples, processing circuitry 126 may include one or more processing circuitry modules for performing each or any combination of the functions described herein.

In some examples, processing circuitry 126 may be coupled to memory 124, processing circuitry 136 may be coupled to memory 134, and processing circuitry 146 may be coupled to memory 144. Memory 124, as well as memory 134 and 144, may include any volatile or non-volatile media, such as a random-access memory (RAM), read only memory (ROM), non-volatile RAM (NVRAM), electrically erasable programmable ROM (EEPROM), flash memory, and the like. Memory 124, 134, and 144 may be a storage device or other non-transitory medium. Memory 124, 134, and 144 may be used by processing circuitry 126, 136, and 146, respectively, for example, to store information corresponding MPS handheld device 102 measurements. In some examples, processing circuitry 126, 136, and 146 may store measurements or previously received data in memory 124, 134, and 144, respectively, and/or calculated values for later retrieval. In some examples, MPS handheld device 102 may be powered by a wall-plug, e.g., via alternating current (AC) power and may include power circuitry such as an AC adapter. In some examples, MPS handheld device 102 may be alternatively and/or additionally powered by batteries, solar cells, or any other suitable power source.

Processing circuitry 126 may be coupled to a user interface including a display, user inputs, and outputs. In some examples, the display may include one or more display devices (e.g., monitor, personal digital assistant (PDA), mobile phone, tablet computer, any other suitable display device, or any combination thereof). For example, the display may be configured to display measurements, measurement information, and/or diagnosis information. In some examples, the user input is configured to receive input from a user, e.g., information corresponding to MPS handheld device 102, a patient, and/or a sample, e.g., sample vial 108. For example, a user may input information such as MPS handheld device 102 parameters or sample vial 108 information by manually entering the product number from MPS handheld device 102 or sample vial 108, or by scanning a quick response (QR) code/bar code from MPS handheld device 102 or sample vial 108. In some examples, MPS handheld device 102 and/or sample vial 108 may be labeled with a serial number as well as a QR code or bar code for a user to input information before testing.

The user input may include components for interaction with a user, such as a keypad and a display, which may be the same as the display. In some examples, the display may be a cathode ray tube (CRT) display, a liquid crystal display (LCD) or light emitting diode (LED) display and the keypad may take the form of an alphanumeric keypad or a reduced set of keys associated with particular functions. The user input, additionally or alternatively, may include a peripheral pointing device, e.g., a mouse, via which a user may interact with the user interface. In some examples, the displays may include a touch screen display, and a user may interact with the user input via the touch screens of the displays. In some examples, the user may also interact with the user input remotely via a networked computing device.

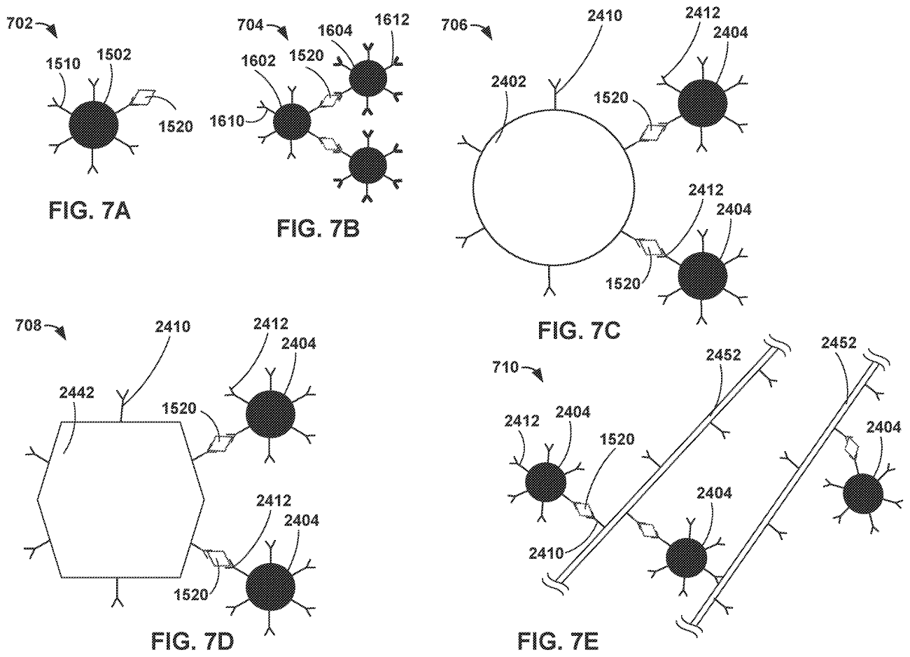

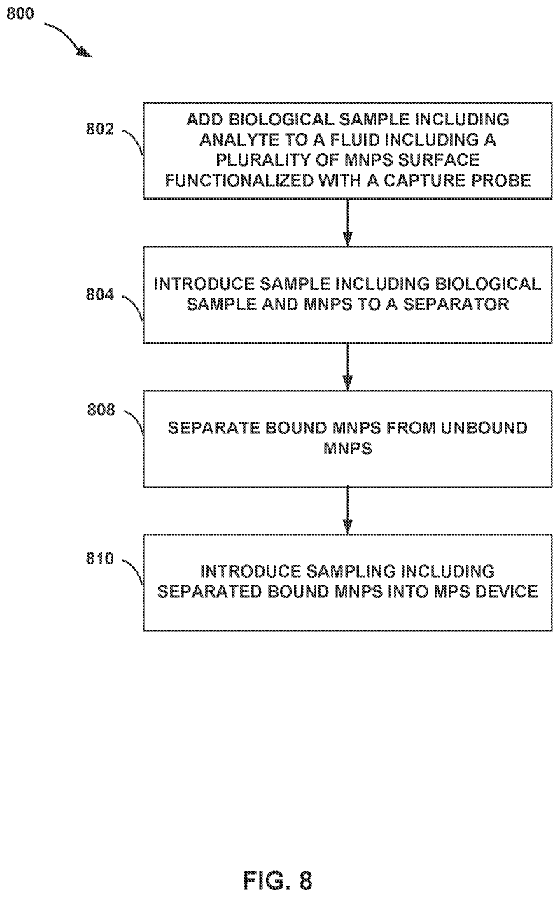

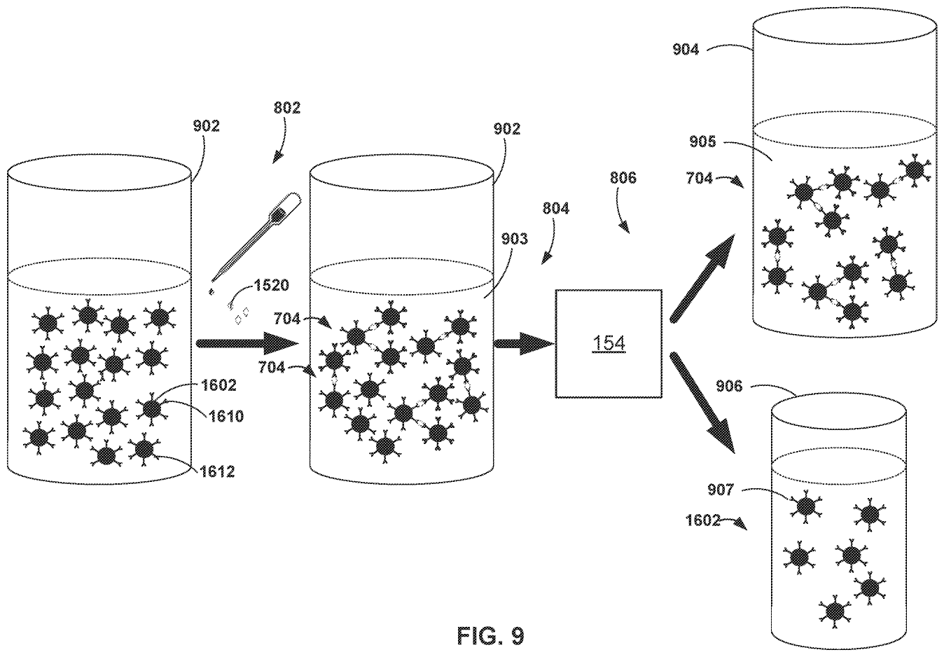

Separator 154 is configured to separate a bound MNP from an unbound MNP. For example, separator 154 is configured to separate any of bound MNPs 702-710 (FIGS. 7A-7D) from any of unbound MNPs 1502, 1602, 1604, 1702, 1802, 1902, 2002, 2004, 2102, 2202, 2204, 2302, 2404, 2504, 2530, or 2552 (FIGS. 8-25) described below. The bound MNPs include a surface functionalized MNP bound to an analyte and may form clusters of MNPs bound to one or more analyte, and the unbound MNP includes a surface functionalized MNP not bound to an analyte. A sample including a fluid comprising at least one bound MNP separated from, and not including, at least one unbound MNP, e.g., sample fluid 903 (FIGS. 9-14) described below, may then be positioned (e.g., within a sample vial 108) in the sample port 110 of MPS device 102 for measurement, e.g., to sense the magnetic response of the sample to an alternating magnetic field. In some examples, separator 154 is configured to separate bound MNPs from unbound MNP by at least one of a filter, an acoustic wave or surface acoustic wave, a gravitation force, a centrifugal force, or a magnetic force.

In some examples, a magnetic response of the sample fluid 903 comprises a Néel relaxation response, e.g., a Néel relaxation response of the sample fluid 903 including a higher order harmonic amplitude response that is greater than a higher order harmonic amplitude response of a Brownian relaxation response of the sample fluid 903. In some examples, the magnetic response of the sample fluid 903 comprises a plurality of higher order harmonics, and a ratio of an amplitude of a first higher order harmonic to a second higher order harmonic is proportional to the binding of analytes to MNPs.

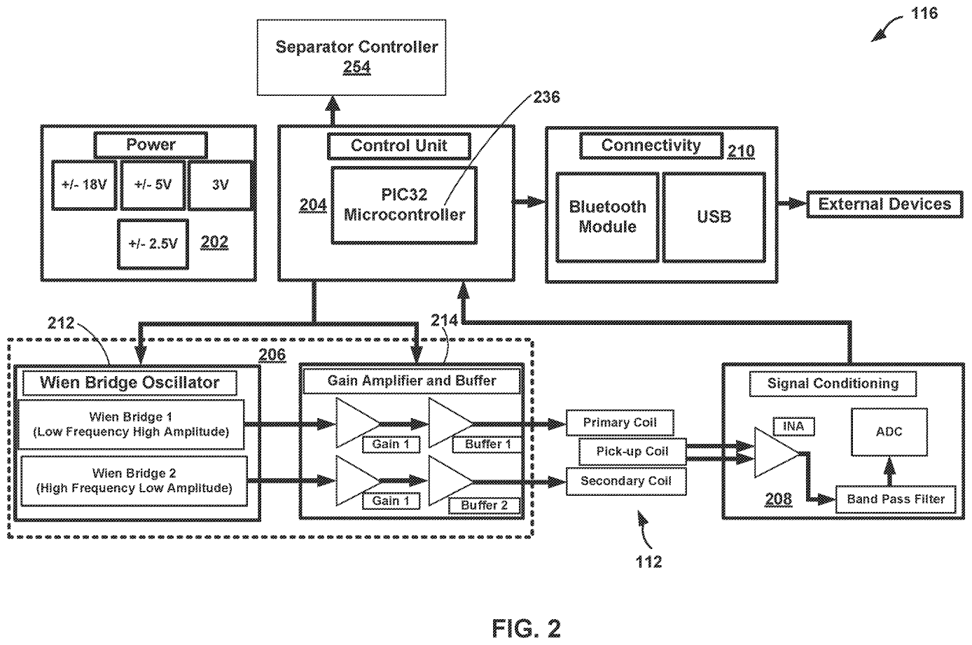

FIG. 2 is a block diagram of example circuitry 116 of an example MPS handheld device 102, in accordance with one or more techniques of this disclosure. In the example shown, circuitry 116 includes power unit 202, control unit 204, coil driver 206, signal conditioning unit 208, connectivity unit 210, coils 112, and separator controller 254.

In the examples shown, power unit 202 is configured to generate electrical power, for example, usable direct current (DC) voltages, from an off-board alternating current (AC) power supply to be used by multiple digital and analog system components. Power unit 202 may comprise an off-board AC to DC power supply adapter to provide DC power to MPS handheld device 102. In some examples, the DC voltage may be further dropped down using high current rated linear drop-off (LDO) regulators and switching power supply components for providing a stable supply at suitable voltages to be used by different stages of MPS handheld device 102. For example, an LDO may be utilized to generate a +/−2.5V supply voltage, or a +/−5V supply voltage, or a +3.3V supply voltage, or any other suitable supply voltage to power an onboard microcontroller. In some examples, switching regulators may be used to generate −15V and +15V supply voltages to feed the coils 112, as described further below. In other examples, power unit 202 may include a battery or other portable power source and associated voltage regulation circuitry.

In some examples, control unit 204 may include microcontroller 236. In some examples, microcontroller 236 may include built-in floating-point hardware and may be utilized as an on-board processor, e.g., as processing circuitry 136. In some examples, microcontroller 236 may communicate with an analog-to-digital converter (ADC) and a communication connection module, e.g., using a using a serial peripheral interface (SPI) protocol. Microcontroller 236 may be additionally configured to select variable frequencies for coils 112, e.g., the primary and secondary drive coils, via SPI protocol to communicate with digital potentiometers for selecting appropriate excitation frequencies.

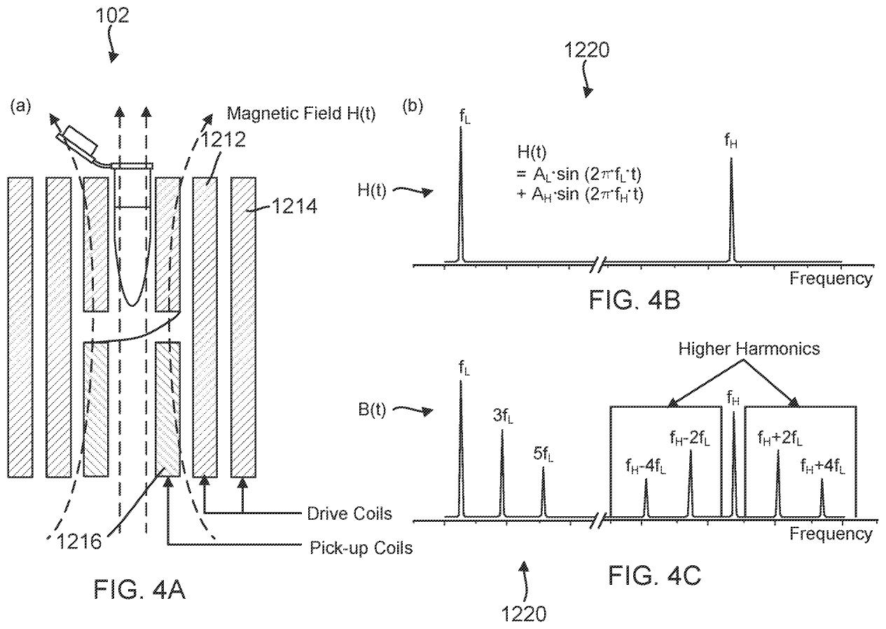

In the example shown, coil driver 206 may be configured to generate variable frequency waveforms for driving coils of coils 112. For example, coil driver 206 may generate two waveforms for primary and secondary drive coil excitation. In some examples, coil driver 206 may generate variable frequency waveforms via two sub-stage units, e.g., Wien-Bridge Oscillator 212 and gain amplifier and buffer 214. Wien-Bridge Oscillator 212 may generate base waveform signals to be further processed by later stages. Gain amplifier and buffer 214 may amplify incoming waveforms and may provide a buffer to meet high current requirements of coils 112. In some examples, waveforms may include any of a sinusoidal waveform, a rectangular or square waveform, a triangular waveform, a sawtooth waveform, or any combination thereof. In some examples, Wien-Bridge Oscillator 212 and gain amplifier and buffer 214 may include corresponding sets of circuitries for generating a low frequency high amplitude waveform for output to a primary coil and for generating a high frequency low amplitude waveform for output to a secondary coil. For example, MPS handheld device 102 may include primary coils 112 (e.g., which may be drive coils 1212 illustrated and described below with reference to FIG. 4A) comprising 1278 turns of 13 American Wire Gage (AWG) copper wire for the low frequency high amplitude waveform, secondary coils 112 (e.g., drive coils 1214 of FIG. 4A below) comprising 449 turns of 30 AWG copper wire for the high frequency low amplitude waveform, and pick-up coils 112 (e.g., pick-up coils 1216 of FIG. 4A below) comprising 36AWG copper wire.

In some examples, coil driver 206 driving coils of coils 112 may be configured to generate phase stable magnetic fields.

In the example shown, signal conditioning unit 208 may be configured to remove (e.g., filter) noise and amplify a signal received from pick-up coils of coils 112. For example, one or more pick-up coils of coils 112 may generate a differential voltage output, and signal conditioning unit 208 may condition the differential voltage output to remove noise and amplify the differential output. In some examples, signal conditioning unit 208 may provide an initial gain and convert the differential signal from pick-up (e.g., search) coils to a single-ended signal for further processing by filtering stages. In some examples, signal conditioning unit 208 may be configured to provide Sallen-Key based second-order low-pass and high-pass filtering to remove the powerline and high-frequency noises. In some examples, a cutoff frequency of a band-pass filter may be set at or near 53 kilohertz (kHz) for the low pass filtering, e.g., as the high frequency cutoff, and at or near 730 Hz for high pass filtering, e.g., as the low frequency cutoff. The filtered signal may be sampled at, for example, 200 kilo-samples per second (ksps) having 16-bit samples via an analog-to-digital (ADC) in communication with microcontroller 236 using SPI protocol. In some implementations, microcontroller 236 may transmit data sampled by the ADC via connectivity unit 210.

In the example shown, connectivity unit 210 may be configured to transfer data via a wired or wireless connection, e.g., connection 118. In some examples, connectivity unit 210 may communicate using any wired or wireless communication modality, for example, serial, universal serial bus (USB), WiFi, local area network (LAN), Bluetooth®, and the like. In some examples, connectivity unit 210 may be configured to execute application software. For example, application software may include a user interface configured to initiate execution of processing of information via microcontroller 236 and display of the processed information in real-time. Application software may further be configured to guide users on how to use MPS handheld device 102. In some examples, the application software may be executed on an external device, for example, computing device 104 or distributed computing system 106, and may use information transferred from connectivity unit 210. In some examples, the application software may be compatible with one or more operating systems, e.g., Windows, iOS®, Android™, or any other suitable operating system.

In some examples, application software may include a mobile application, and may implement a Fast Fourier Transform (FFT) for frequency-domain processing of incoming information, e.g., executed by processing circuitry 126, 136, and/or 146. In some examples, FFT implementation in the mobile application may be executed and provide results within seconds. In some examples, FFT implementation may result in information such as frequency harmonic amplitudes and phase information, and may be used to derive immunoassay detection. In some implementations, harmonic amplitudes, phase angle information, and harmonic ratios may be metrics for quantifying target analytes from testing samples, such as sample vial 108.

Separator controller 254 may be configured to control separator 154. In the example shown, microcontroller 236 is configured to communicate with separator controller 254, e.g., in examples in which separator 154 is integrated into MPS handheld device 102. Separator controller 254 may comprise processing circuitry, and may be substantially similar to processing circuitry 136, or may be an example of processing circuitry 136. In other examples, separator controller 254 may be processing circuitry that is separate from, and may or may not be in communication with microcontroller 236, e.g., in examples in which separator 154 is separate from MPS handheld device 102.

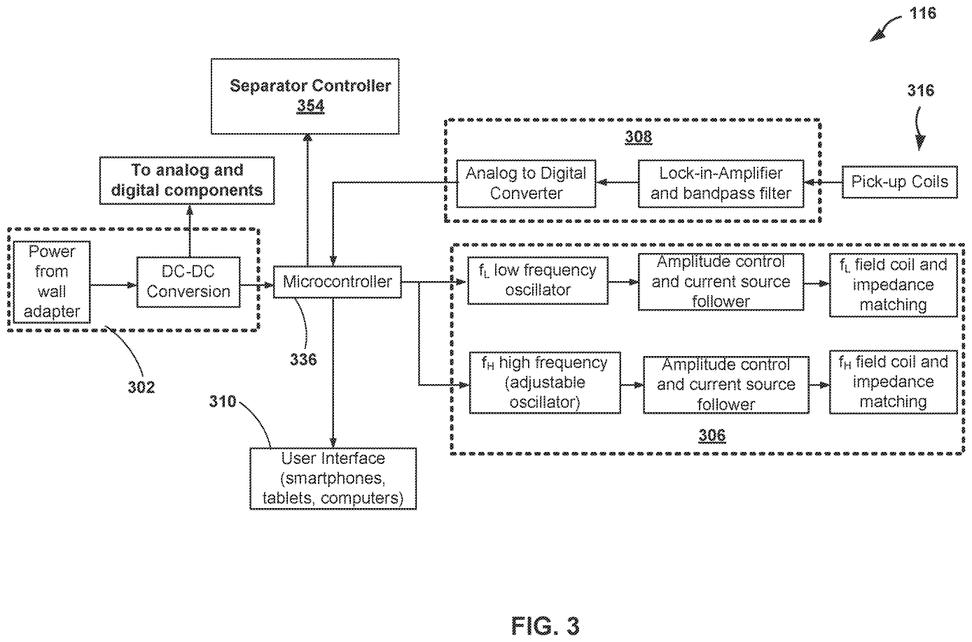

FIG. 3 is a block diagram of another example circuitry 116 of an example MPS handheld device 102, in accordance with one or more techniques of this disclosure. In the example shown, circuitry 116 includes power unit 302, microcontroller 336, coil driver 306, signal conditioning unit 308, connectivity unit 310, coils 112, and separator controller 354.

In the examples shown, power unit 302 is configured to generate electrical power, for example, usable direct current (DC) voltages from an off-board alternating current (AC) power supply, e.g., a wall outlet. Power unit 302 may be substantially similar to power unit 202 illustrated and described above with respect to FIG. 2, and may additionally include DC to DC conversion.

Microcontroller 336 may be utilized as an on-board processor, e.g., as processing circuitry 136, and may be substantially similar to microcontroller 236 illustrated and described above with respect to FIG. 2. Separator controller 354 may be configured to control separator 154, and may be substantially similar to separator controller 254 illustrated and described above with respect to FIG. 2.

In the example shown, coil driver 306 may be configured to generate variable frequency waveforms for drive coils, e.g. coils 112, and may be substantially similar to coil driver 206 illustrated and described above with respect to FIG. 2. In the examples shown, coil driver 306 may generate a low frequency waveform via a low frequency oscillator and a high frequency waveform via a high frequency oscillator. Coil driver 306 may be configured to control the amplitudes and impedance matching of the low and high frequency waveforms. In some examples, waveforms may include any of a sinusoidal waveform, a rectangular or square waveform, a triangular waveform, a sawtooth waveform, or any combination thereof.

In the example shown, signal conditioning unit 308 may be configured to remove noise and amplify a signal received from pick-up coils 316, and perform analog to digital conversion. In some examples, signal conditioning unit 308 may be substantially similar to signal conditioning unit 208. For example, one or more pick-up coils of coils 112 may generate a differential voltage output, and signal conditioning unit 308 may operate to condition the differential voltage signal similar to signal conditioning unit 208 illustrated and described above with respect to FIG. 2.

FIGS. 4A-6C illustrate one or more example operating principles of an MPS device, for example, MPS handheld device 102, and will be described concurrently below.

FIG. 4A is a schematic cross-sectional illustration of a portion of an example MPS handheld device 102, in accordance with one or more techniques of this disclosure. In the example shown, sample vial 108 is in position within sample loading port 110 in MPS handheld device 102 during a measurement, e.g., while drive coils 1212 and 1214 generate an alternating magnetic field H(t) proximate sample vial 108 and pick-up coils 1216 detect the resulting magnetic responses from MNPs within sample vial 108. Pick-up coils 1216 may be designed to have half of its portion clockwise wound and the other half portion counter-clockwise wound. This design removes the signal caused by alternating magnetic field H(t) and allows the pick-up coils 1216 to specifically detect the magnetic responses of MNPs. In some examples, drive coils 1212 may generate a magnetic field having a low frequency, f_{L}, relative to a magnetic field generated by drive coils 1214 having a higher frequency, f_{H} based on drive signals generated by processing circuitry of MPS handheld device 102. Each of drive coils 1212 and 1214 may additionally generate magnetic fields having different amplitudes, e.g., A_{L} generated via low frequency drive coils 1212 and A_{H} generated via high frequency drive coils 1214. The magnetic fields generated by drive coils 1212 and 1214 may be in phase, and may add via superposition, resulting in generation of the composite magnetic field H(t). In some examples, drive coils 1212 may be wound in an opposite direction from drive coils 1214, e.g., counter-clockwise for drive coils 1212 and clockwise for drive coils 1214, or clockwise for drive coils 1212 and counter-clockwise for drive coils 1214. In some examples, drive coils 1212 and 1214 may be wound in the same direction.

FIG. 4B is an illustration of an example plot 1220 of the amplitude of composite magnetic field H(t) as a function of frequency, in accordance with one or more techniques of this disclosure. In the example shown, drive coils 1212 and 1214 each generate a sinusoidal magnetic field, and plot 1220 illustrates the frequency content of composite magnetic field H(t), namely two substantially single frequency spikes, or delta functions, at f_{L} and f_{H} and having amplitudes A_{L} and A_{H}, respectively.

FIG. 4C is an illustration of an example plot 1250 of the amplitude of magnetic flux B(t) due to the magnetic responses of MNPs detected by pick-up coils 1216 as a function of frequency, in accordance with one or more techniques of this disclosure. In the example shown, the MNPs within sample vial 108 respond to magnetic field H(t), which may generate harmonics of H(t) that are detected by pick-up coils 1216. In the example shown, the magnetic flux B(t) including magnetic responses of MNPs may include several harmonic frequencies of varying amplitudes at various frequencies.

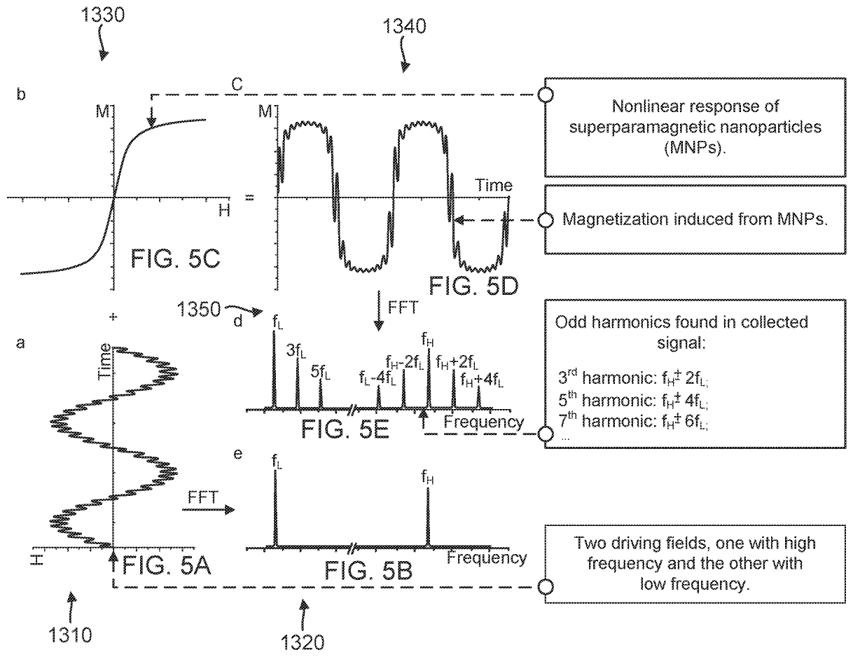

FIGS. 5A-5E illustrate the operating principle of FIGS. 4A-4C in more detail.

FIG. 5A is an illustration an example plot 1310 of the amplitude of an alternating magnetic field H(t) generated by drive coils as a function of time, in accordance with one or more techniques of this disclosure. For example, drive coils 1212 and 1214 may generate sinusoidal magnetic fields, and FIG. 5A illustrates the superposition of each low and high frequency sinusoidal magnetic field generated by drive coils 1212 and 1214, along with their corresponding differing amplitudes. FIG. 5B is an illustration an example plot 1320 of the amplitude of composite magnetic field H(t) as a function of frequency, in accordance with one or more techniques of this disclosure. In the example shown, FIG. 5B illustrates the spectral (temporal) content of H(t) of FIG. 5A, similar to FIG. 4B described above.

FIG. 5C is an illustration of an example plot 1330 of the magnetic response of MNPs to static magnetic fields (not alternating magnetic field), in accordance with one or more techniques of this disclosure. In the example shown, MNPs may by superparamagnetic, and may have a nonlinear response to an applied magnetic field.

FIG. 5E is an illustration of an example plot 1350 of the amplitude of the alternating magnetic responses of FIG. 5D as a function of frequency, in accordance with one or more techniques of this disclosure. FIG. 5D explicitly illustrates the additional harmonic content generated by the magnetic response of MNPs within sample vial 108.

In some examples, the magnetic response of MNPs within sample vial 108 may change based on the presence of analytes in a biofluid added to sample vial 108. For example, the analytes may bind to the surface functionalized MNPs and alter the magnetic response of the MNPs relative to the magnetic response of surface functionalized MNPs with no analytes present in the biofluid. In some examples, the altered magnetic response of the MNPs due to analytes may be distinguished from features of detected magnetic flux B(t), for example, via changes to the amplitudes and/or frequencies of the spectral content, e.g., harmonics, of B(t).

For example, in the presence of oscillating magnetic fields, MNPs may be magnetized and their magnetic moments may tend to align with the magnetic fields. For a ferrofluid system of monodispersed, noninteracting MNPs, the magnetic response may obey a Langevin function:

$${M}_{D} ( t ) = {m}_{s} cL ( ξ ) ,$$

$$where ,$$

$$L ( ξ ) = coth ξ - \frac{1}{ξ} , ξ = \frac{{m}_{s} H ( t )}{{k}_{B} T}$$

The MNPs are characterized by magnetic core diameter D, saturation magnetization Ms and concentration c. In some examples, MNPs may be assumed to be spherical and without mutual interactions. Consequently, the magnetic moment of each particle may be mₛ=MsπD³/6, where V_{c}=πD³/6 is the volume of the magnetic core, ξ is the ratio of magnetic energy over thermal energy, k_{B} is Boltzmann's constant, and T is the absolute temperature in Kelvin. The external magnetic fields may be expressed as H(t)=A_{H} cos(2πf_{H}t)+A_{L} cos(2πf_{H}t) where A_{H}, A_{L}, f_{H}, and f_{L} are the amplitude and frequency of high and low frequency fields, respectively.

The harmonics generated by MNPs at specific frequencies may be represented by a phasor, e.g., Ae^{j(ωt+φ)}, or A∠φ, where ω is the angular frequency of the driving field, A is the harmonic amplitude, φ is the harmonic phase, and j is the square root of negative one.

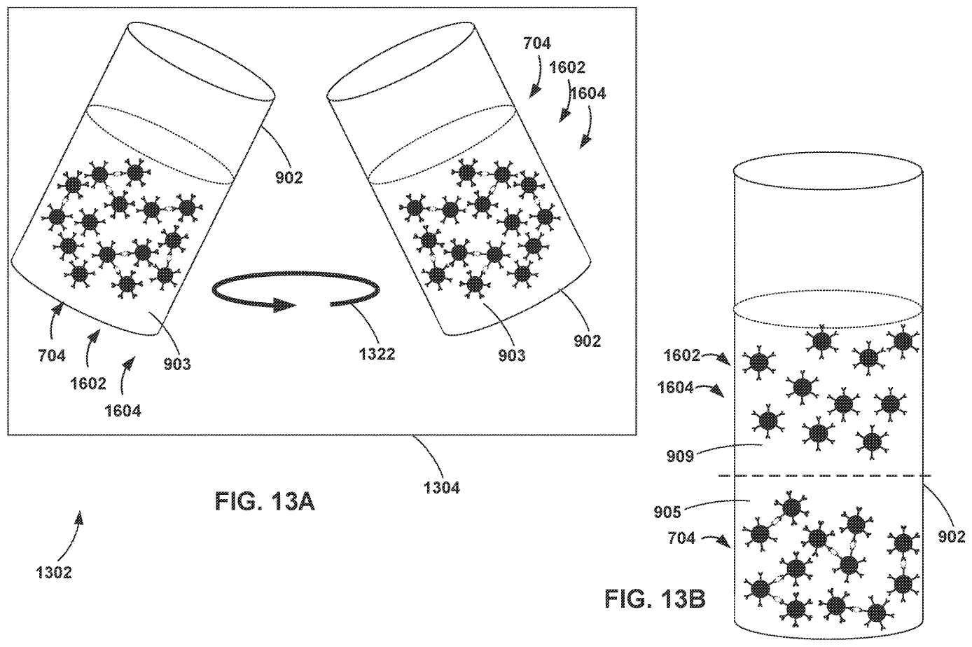

FIG. 5D is an illustration of an example plot 1340 of the amplitude of an alternating magnetic responses of MNPs detected by pick-up coils 1216 as a function of time, in accordance with one or more techniques of this disclosure. The example shown in FIG. 13D illustrates the effects of the MNPs on the applied magnetic field, e.g., perturbation of H(t) resulting in generation of harmonics.

According to Faraday's law, the induced voltage in a pair of pick-up coils is expressed as:

$$u ( t ) = - {S}_{0} V \frac{d}{dt} {M}_{D} ( t )$$

where V is the volume of an MNP suspension. Pick-up coil sensitivity S₀ is equal to the external magnetic field strength divided by current.

Taylor expansion of M_{D}(t) shows the major frequency mixing components:

$$\frac{{W}_{D} ( t )}{{m}_{s} c} = L ( \frac{{m}_{s} H ( t )}{{k}_{B} T} ) = \frac{1}{3} ( \frac{{m}_{s}}{{k}_{B} T} ) H ( t ) - \frac{1}{45} {( \frac{{m}_{s}}{{k}_{B} T} )}^{3} {H ( t )}^{3} + \frac{2}{945} {( \frac{{m}_{s}}{{k}_{B} T} )}^{5} {H ( t )}^{5} + … = … + [ - \frac{1}{60} {A}_{H} {{A}_{L}^{2} ( \frac{{m}_{s}}{{k}_{B} T} )}^{3} + … ] \times cos [ 2 π ( {f}_{H} \pm 2 {f}_{L} ) t + {φ}_{{f}_{H} \pm 2 {f}_{L}} ] + [ \frac{1}{1512} {A}_{H} {{A}_{L}^{2} ( \frac{{m}_{s}}{{k}_{B} T} )}^{5} + … ] \times cos [ 2 π ( {f}_{H} \pm 4 {f}_{L} ) t + {φ}_{{f}_{H} \pm 4 {f}_{L}} ] + …$$

The mixing frequency components are found at odd harmonics exclusively:

$${{M}_{D} ( t ) \vert}_{3 rd} \approx \frac{- {m}_{s} c}{60} {A}_{H} {{A}_{L}^{2} ( \frac{{m}_{s}}{{k}_{B} T} )}^{3} \times cos [ 2 π ( {f}_{H} + 2 {f}_{L} ) t + {φ}_{{f}_{H} \pm 2 {f}_{L}} ]$$

$${{M}_{D} ( t ) \vert}_{5 th} \approx \frac{{m}_{s} c}{1512} {A}_{H} {{A}_{L}^{2} ( \frac{{m}_{s}}{{k}_{B} T} )}^{5} \times cos [ 2 π ( {f}_{H} + 4 {f}_{L} ) t + {φ}_{{f}_{H} \pm 4 {f}_{L}} ]$$

Amplitudes of induced voltages at the 3^{rd} and 5^{th} harmonics may be expressed as:

$${{u}_{3 rd} = - {S}_{0} V \frac{d}{dt} {M}_{D} ( t ) \vert}_{3 rd}$$

$${{u}_{5 th} = - {S}_{0} V \frac{d}{dt} {M}_{D} ( t ) \vert}_{5 th}$$

The harmonic amplitudes of the 3^{rd} and 5^{th} harmonics may be simplified as:

$${A}_{3 rd} ∝ ( {f}_{H} \pm 2 {f}_{L} ) \times cos [ {φ}_{{f}_{H} \pm 2 {f}_{L}} ]$$

$${A}_{5 th} ∝ ( {f}_{H} \pm 4 {f}_{L} ) \times cos [ {φ}_{{f}_{H} \pm 4 {f}_{L}} ]$$

$$where$$

$${φ}_{{f}_{H} \pm 2 {f}_{L}} = arc tan ( 2 π ( {f}_{H} \pm 2 {f}_{L} ) {τ}_{eff} )$$

For iron oxide MNPs with diameters of 20 nanometers (nm), the effective relaxation time is dominated by Brownian relaxation:

$$τ = {τ}_{eff} = {τ}_{B 0} = \frac{3 η {V}_{h}}{{k}_{B} T}$$

In some examples, a change in MNP hydrodynamic size may cause a change in harmonic angle (phase angle), which further may cause a change in harmonic amplitude. Harmonic amplitudes may be proportional to the number of MNPs in a testing vial, and to make each testing result repeatable, a harmonic ratio of the 3^{rd} harmonic over the 5^{th} harmonic may be used to reduce and/or eliminated the effect of MNP quantities in the testing vial. The harmonic ratio of the 3^{rd} over the 5^{th} harmonics may be expressed as:

$$R 35 = \frac{{u}_{3 rd}}{{u}_{5 th}} = \frac{{\frac{d}{dt} {M}_{D} ( t ) \vert}_{3 rd}}{{\frac{d}{dt} {M}_{D} ( t ) \vert}_{5 th}} = \frac{( {f}_{H} \pm 2 {f}_{L} ) \times cos [ {φ}_{{f}_{H} \pm 2 {f}_{L}} ]}{( {f}_{H} \pm 4 {f}_{L} ) \times cos [ {φ}_{{f}_{H} \pm 4 {f}_{L}} ]}$$

In some examples, because f_{L}<<f_{H}, the harmonic ratio of the 3^{rd} over the 5^{th} may be further simplified as:

$$R 35 = \frac{cos [ {φ}_{{f}_{H} \pm 2 {f}_{L}} ]}{cos [ {φ}_{{f}_{H} \pm 4 {f}_{L}} ]}$$

In some examples, a change in MNP hydrodynamic size may cause a change in harmonic angle, which may further cause a change in the harmonic amplitude ratio.

Harmonic ratio may be used as an MNP quantity-independent parameter to monitor the binding of target analytes onto MNPs, e.g., the hydrodynamic size change. In addition, any kinds of harmonic ratios such as R37 (the 3rd over the 7th harmonic ratio), R57 (the 5th over the 7th harmonic ratio), Rij (the ith over the jth harmonic ratio, where i and j are odd numbers and i≠j), or any other harmonic ratios, may be used.

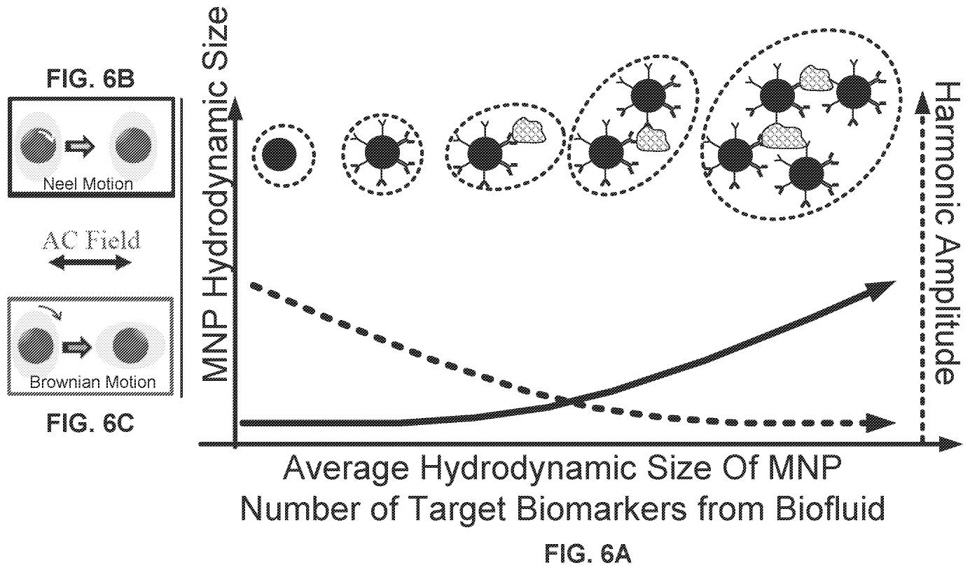

FIG. 6A is an illustration of an example plot of the harmonic amplitude response as a function of hydrodynamic size of MNPs (or the number of target biomarkers from a biofluid, e.g., analytes of a biological sample), in accordance with one or more techniques of this disclosure. In the example shown, as the degree of MNP self-assembly increases in the presence of one or more analytes, the average hydrodynamic size of MNPs and MNP self-assemblies, e.g., clusters, increases, and the measured harmonic amplitudes decrease.

FIGS. 6B and 6C are illustrations of an example Neel and Brownian motions of an MNP, respectively, in accordance with one or more techniques of this disclosure. For example, as shown in FIG. 6B, Neel relaxation or motion includes reorientation of a magnetization vector inside an MNP, e.g., inside the magnetic core against an energy barrier. As shown in FIG. 6C, Brownian relaxation or motion is due to rotational diffusion of a whole particle, e.g., an MNP, or a cluster of MNPs.

FIGS. 7A-7E illustrate example bound MNPs 702-710 having hydrodynamic sizes that are larger than unbound MNPs and may be detectable via MPS, for example, by using MPS handheld device 102. In some examples, the bound MNPs 702-710 may be separable from unbound MNPs, e.g., via the methods and devices described herein. In some examples, bound MNPs 702-710 may be MNP test materials, e.g., for use with MPS device 102.

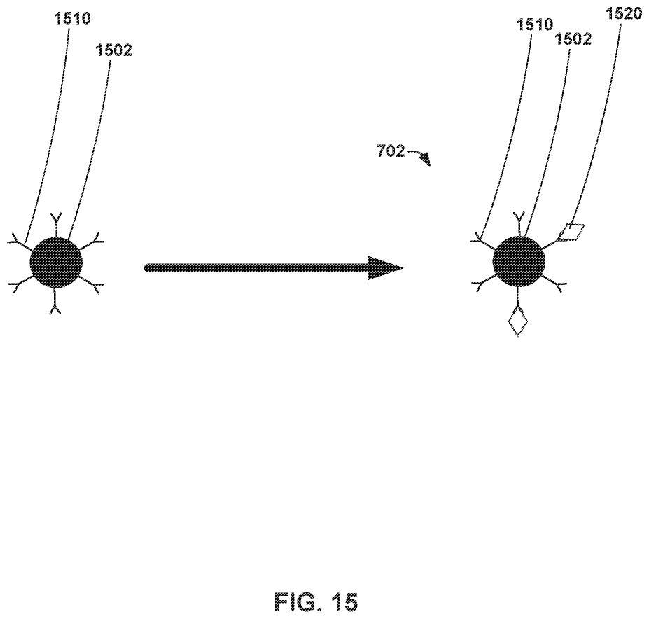

FIG. 7A is an illustration of an example bound MNP 702, in accordance with one or more techniques of this disclosure. In the example shown, bound MNP 702 may include MNP 1502 that may be surface functionalized via a coating of one or more monoclonal antibodies 1510 bound to an antigen 1520 (FIG. 15). Bound MNP 702 may have an increased hydrodynamic size, e.g., relative to an unbound MNP 1502, as a result of the binding of antigens 1520 to monoclonal antibodies 1510. In other examples, bound MNP 702 may comprise any of MNPs 1602, 1604, 1702, 1802, 1902, 2002, 2004, 2102, 2202, 2204, 2302, 2404, 2504, 2530, or 2552 bound to a respective antigen 1520, DNA-binding protein 1820, single strand DNA and/or RNA 1920, heavy metal ion 2120, 2122, and/or 2220, target protease 2320, streptavidin protein 2526, biotinylated antisense oligonucleotide 2560 and/or analyte 2570 by a respective analyte, e.g., monoclonal antibodies 1510, antibodies 1610, 1612, polyclonal antibodies 1710, 1712, and/or 1714, single strand DNA and/or RNA 1810, reverse complementary single strand DNA and/or RNA 1910, single strand DNA and/or RNA 2010 and/or 2012, structure switching strand DNA and/or RNA 2110, single strand DNA and/or RNA 2210 and/or 2212, peptides 2310, antibodies 2410, 2412, single strand DNA and/or RNA 2510 and/or 2512, biotin 2524, biotin 2524 and streptavidin proteins 2526 (e.g., which may form streptavidin-saturated biotinylated MNPs 2554) thereby increasing the hydrodynamic size of the bound MNP 702 relative to unbound MNP 1602, 1604, 1702, 1802, 1902, 2002, 2004, 2102, 2202, 2204, 2302, 2404, 2504, 2530, or 2552, e.g., as described below with reference to FIGS. 15-25C.

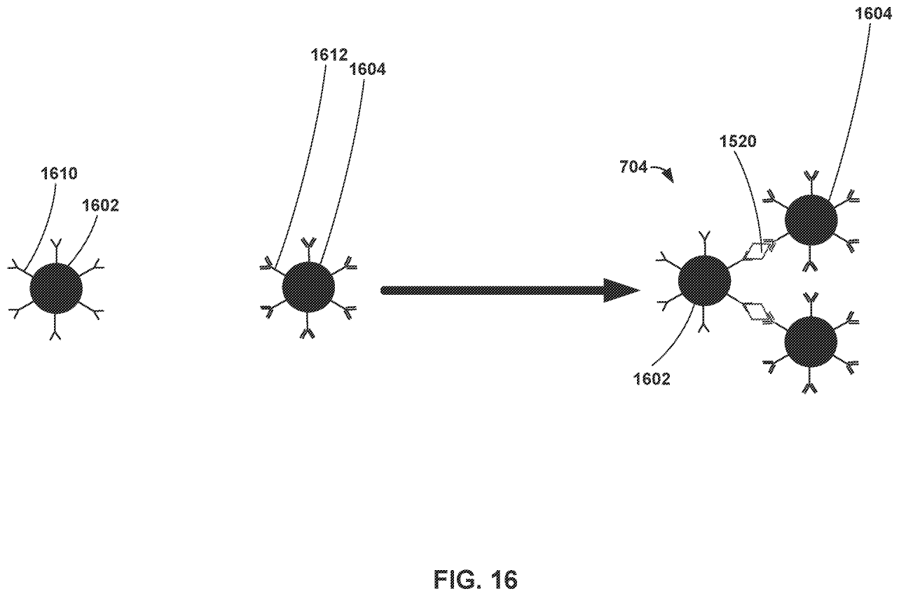

FIG. 7B is an illustration of an example bound MNPs 704, in accordance with one or more techniques of this disclosure. In the example shown, bound MNPs 704 may include a cluster of MNPs, e.g., a plurality of conjugated MNPs. In the example shown, bound MNP 704 includes a plurality of MNPs 1602, 1604 bound via monoclonal antibodies 1510 and antigens 1520 (FIG. 16). Bound MNPs 704 may have an increased hydrodynamic size, e.g., relative to an unbound MNPs 1602, 1604, as a result of the binding of antigens 1520 to monoclonal antibodies 1510 and clustering of MNPs 1602, 1604. In some examples, bound MNP 704 may include a plurality of MNPs that cluster and/or are bound via any suitable probe-analyte and/or antibody-antigen binding scheme. For example, MNPs 602, 1604 may be surface functionalized with polyclonal antibodies 1610, 1612 that bind to different epitopes from target antigen 1520, and may allow cluster formation. In other examples, MNPs 602, 1604 may be surface functionalized with antisense nucleotides (ASOs) that may bind to different locations of target gene (ssDNA, RNA), which may allow cluster formation.

In other examples, bound MNPs 704 may comprise a cluster of a plurality of any of MNPs 1602, 1604, 1702, 1802, 1902, 2002, 2004, 2102, 2202, 2204, 2302, 2404, 2504, 2530, or 2552 bound to a respective antigen 1520, DNA-binding protein 1820, single strand DNA and/or RNA 1920, heavy metal ion 2120, 2122, and/or 2220, target protease 2320, streptavidin protein 2526, biotinylated antisense oligonucleotide 2560 and/or analyte 2570 by a respective analyte, e.g., monoclonal antibodies 1510, antibodies 1610, 1612, polyclonal antibodies 1710, 1712, and/or 1714, single strand DNA and/or RNA 1810, reverse complementary single strand DNA and/or RNA 1910, single strand DNA and/or RNA 2010 and/or 2012, structure switching strand DNA and/or RNA 2110, single strand DNA and/or RNA 2210 and/or 2212, peptides 2310, antibodies 2410, 2412, single strand DNA and/or RNA 2510 and/or 2512, biotin 2524, biotin 2524 and streptavidin proteins 2526 (e.g., which may form streptavidin-saturated biotinylated MNPs 2554) thereby increasing the hydrodynamic size of the bound MNP 704 relative to unbound MNP 1602, 1604, 1702, 1802, 1902, 2002, 2004, 2102, 2202, 2204, 2302, 2404, 2504, 2530, or 2552, e.g., as described below with reference to FIGS. 15-25C.

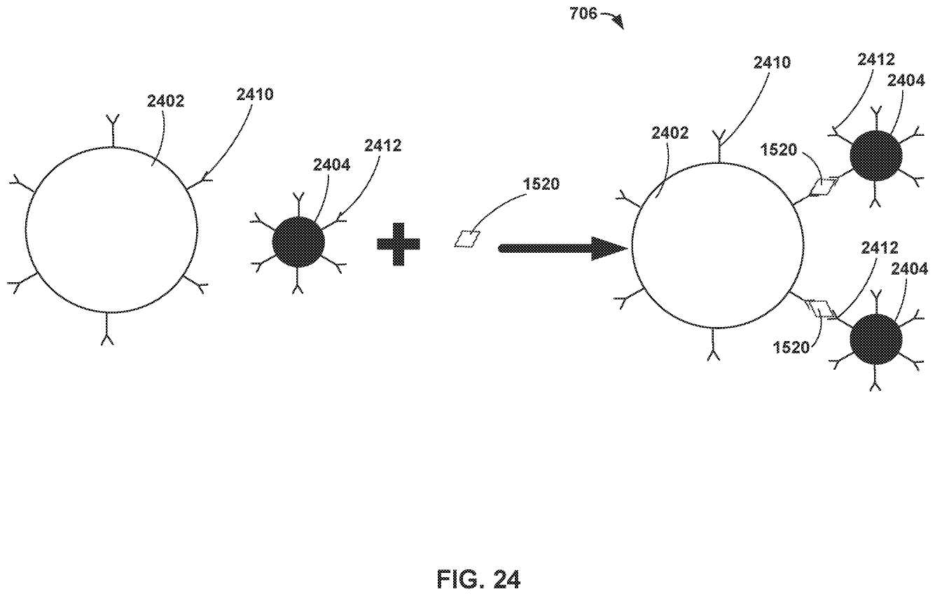

FIG. 7C is an illustration of an example bound MNPs 706, in accordance with one or more techniques of this disclosure. In the example shown, bound MNP 706 may include one or more MNPs bound to a nonmagnetic structure, e.g., a microbead. In the example shown, bound MNP 706 includes a plurality of MNPs 2404 bound to microbead 2402 via antibodies 2410 and 2412 and antigens 1520 (FIG. 24). In some examples, bound MNP 706 may comprise a cluster including a plurality of surface functionalized MNPs bound with the one or more microbeads 2402 via capture of an analyte upon combination with a biological sample including the analyte. Bound MNP 706 may have an increased hydrodynamic size of an MNP, e.g., relative to an unbound MNPs 2404, as a result of the binding of antigens 1520 to monoclonal antibodies 2410, 2412 binding one or more MNPs 2404 to microbead 2402. In some examples, antibodies 2410, 2412 may be monoclonal detection and/or capture antibodies, and cluster formation between one or more microbeads 2402 and one or more MNPs 2404 may be facilitated by specific antibody-antigen reactions following a sandwich bioassay mechanism. In some examples, MNPs 2404 and microbeads 2402 may be surface functionalized with capture DNA probes and detection DNA probes, respectively. For example, each probe may be complementary to one end of a target gene, which may allow cluster formation with one or more microbeads 2402 and one or more MNPs 2404, e.g., formation of bound MNP 706. In some examples, microbead 2402 is configured to increase a surface area of MNP 2404, e.g., to form bound MNP 706 having a surface area larger than that of an unbound MNP.

In other examples, bound MNP 706 may comprise any suitable microbead (e.g., microbeads 2502) bound to any of the MNPs described here, e.g., MNPs 1602, 1604, 1702, 1802, 1902, 2002, 2004, 2102, 2202, 2204, 2302, 2504, 2530, or 2552 bound to any of the analytes described herein, e.g., antigen 1520, DNA-binding protein 1820, single strand DNA and/or RNA 1920, heavy metal ion 2120, 2122, and/or 2220, target protease 2320, streptavidin protein 2526, biotinylated antisense oligonucleotide 2560 and/or analyte 2570 by any of the analytes described herein, e.g., monoclonal antibodies 1510, antibodies 1610, 1612, polyclonal antibodies 1710, 1712, and/or 1714, single strand DNA and/or RNA 1810, reverse complementary single strand DNA and/or RNA 1910, single strand DNA and/or RNA 2010 and/or 2012, structure switching strand DNA and/or RNA 2110, single strand DNA and/or RNA 2210 and/or 2212, peptides 2310, antibodies 2410, 2412, single strand DNA and/or RNA 2510 and/or 2512, biotin 2524, biotin 2524 and streptavidin proteins 2526 (e.g., which may form streptavidin-saturated biotinylated MNPs 2554) thereby increasing the hydrodynamic size of the bound MNP 706 relative to unbound MNP 1602, 1604, 1702, 1802, 1902, 2002, 2004, 2102, 2202, 2204, 2302, 2404, 2504, 2530, or 2552, e.g., as described below with reference to FIGS. 15-25C. Microbead 2402 may be comprised of silicon, gold, a polymer or plastic, or any suitable non-magnetic material.

FIG. 7D is an illustration of an example bound MNPs 708, in accordance with one or more techniques of this disclosure. Bound MNP 708 may be substantially similar to bound MNP 706, except for the differences described herein. For example, bound MNP 708 may include microstructure 2442 rather than microbead 2402. Microstructure 2442 may be substantially similar to microbead 2402, except that microstructure 2442 may have a different shape, e.g., a regular or irregular shape. In some examples, microstructure 2442 may be nonmagnetic and/or may be a microparticle, microcube, or the like. Microstructure 2442 may have a non-spherical regular or irregular shape, e.g., a polygonal or non-polygonal and non-spherical shape. In some examples, the non-spherical shape of microstructure 2442 may improve binding of probes to analytes and/or separation of bound MNP 708 from unbound MNPs relative to a spherical bound MNP 706 by one or more of the separation methods and device described herein, e.g., with reference to FIGS. 8-14. For example, non-spherical shaped microstructure 2442 may provide an increased size, weight, and/or surface area of bound MNP 708 which may improve separation from unbound MNPs via a larger difference in amount of settling within a fluid due to gravity and/or centrifuge, a larger difference in fluid resistance (friction) to motion induced by acoustic and/or magnetic forces.

FIG. 7E is an illustration of an example bound MNPs 710, in accordance with one or more techniques of this disclosure. Bound MNP 710 may be substantially similar to bound MNP 708, except for the differences described herein. For example, bound MNP 710 may include microrods 2452 rather than microstructure 2442. Microrods 2452 may be substantially similar to microstructure 2442, except that microrods 2452 may have a rod-like shape, e.g., a size in one dimension that is significantly longer than in the other two dimensions. For example, microrods 2452 may have a length that is greater than or equal to twice its width and/or height.

In some examples, microrods 2452 may be nonmagnetic. In some examples, the rod-like shape of microrods 2452 may improve binding of probes to analytes and/or separation of bound MNP 708 by providing a larger cross-sectional surface, especially when clustered. For example, microrods 2452 may provide a significantly increased cross-sectional area, relative to bound MNPs 702-708 for example, which may improve separation from unbound MNPs via an even larger difference in fluid resistance (friction) to motion.

FIGS. 8-14 illustrate example methods and devices for separating bound MNPs from unbound MNPs. In some examples, the bound MNPs may be one or more of bound MNPs 702-710 separable from unbound MNPs, e.g., separable from any of the unbound MNPs 1502, 1602, 1604, 1702, 1802, 1902, 2002, 2004, 2102, 2202, 2204, 2302, 2404, 2504, 2530, or 2552 disclosed herein.

FIG. 8 is a flowchart of an example method 800 of detecting chemicals and/or biological substances via MPS including separating bound MNPs from unbound MNPs, in accordance with one or more techniques of this disclosure. FIG. 9 is a schematic illustration of steps of method 800, in accordance with one or more techniques of this disclosure, and is described in conjunction with FIG. 8. The example method of FIG. 8 is described with respect to diagnosis system 100 including an MPS handheld device 102 of FIG. 1 and circuitry 116 of FIGS. 1-3B. The example method may be performed, for example, by a user interacting with separator 154, MPS handheld device 102, and a computing device 104 and/or distributed computing device 106, executing the steps of the method. Although described with reference to separator 154, MPS handheld device 102, and a computing device 104, the method of FIG. 8 is not so limited and may be performed with other devices, separators, diagnostic devices (e.g., MPS, MPI, NMR, QD, fluorescent biosensors, or the like), and/or computing devices.

A user and/or a device may add a biological sample, such as a bodily fluid or tissue sample, which may include analytes 1520 to a fluid comprising a plurality of MNPs 1602, 1604 that have been surface functionalized and include capture probes 1610, 1612 (802). In the example shown, analytes 1520 are added to container 902 including a fluid including a plurality of MNPs 1602, 1604. Specific binding of analytes to probes causes MNPs to form bound MNPs and/or clusters of bound MNPs. In the example shown, sample 903 comprises a fluid including bound MNPs 704 and unbound MNPs 1602, 1604, however, sample 903 may include any of bound MNPs 702-710 and unbound MNPs 1502, 1602, 1604, 1702, 1802, 1902, 2002, 2004, 2102, 2202, 2204, 2302, 2404, 2504, 2530, or 2552 described above.

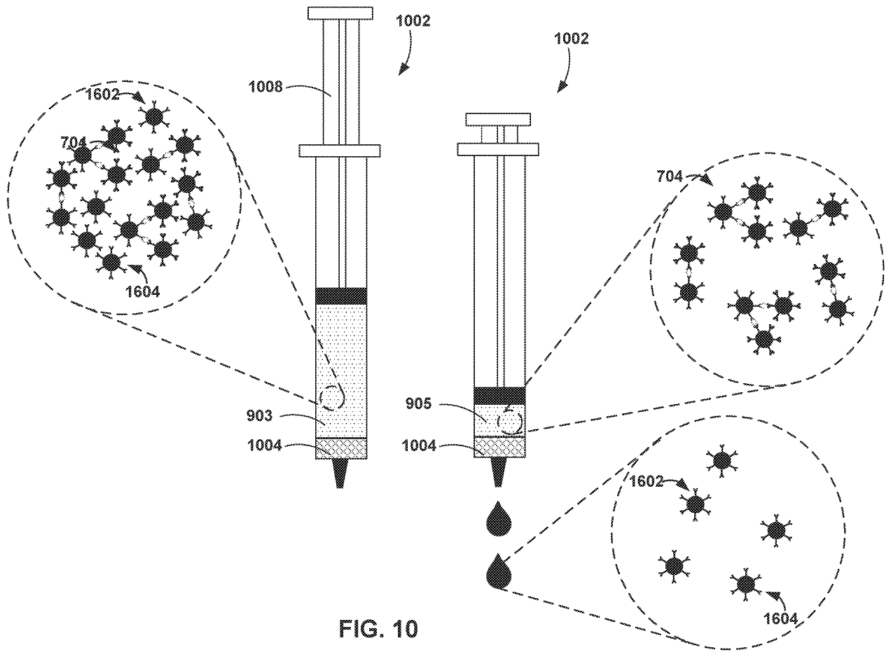

The user and/or device may then introduce the sample 903 to separator 154 (804), and separator 154 may separate bound MNPs 704 from unbound MNPs 1602, 1604 (806). For example, separator 154 may separate bound MNPs 704 from unbound MNPs 1602, 1604 by filtering, gravity and/or sedimentation, centrifuging, magnetic separation, surface acoustic waves (SAW), or any other suitable separation method, e.g., described below with reference to FIGS. 10-14.

After separation, the user and/or device may introduce the separated sample 905 within an MPS device 102 (808). For example, after separation, the bound MNPs 704 may be within a sample fluid 905 within container 904 and the user may place at least a portion of the sample fluid 905 within a sample port of MPS device 102. The unbound MNPs 1602 may be within a sample fluid 907, and the user discard and/or reclaim unbound MNPs 1602, 1604 in container 906.

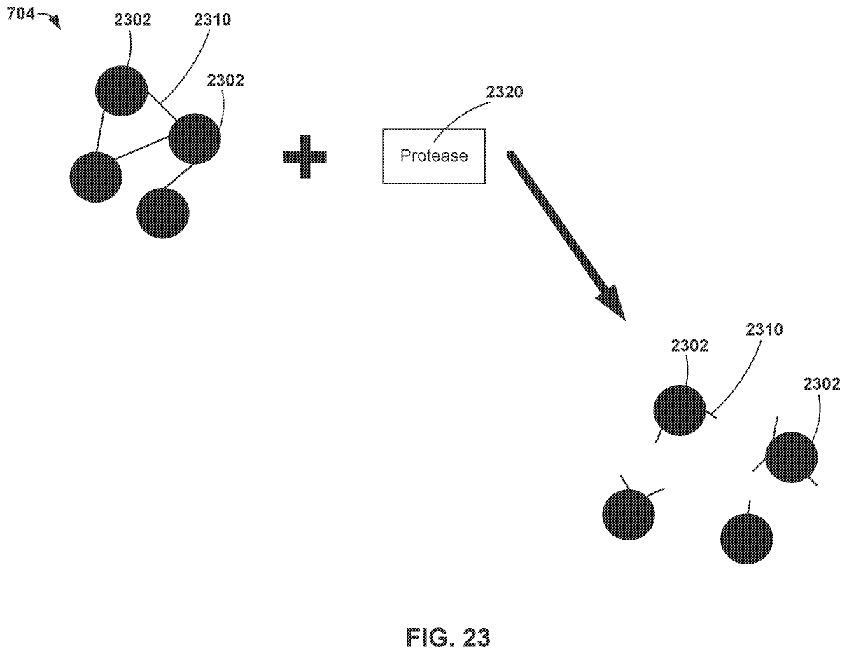

In some examples, the user may place at least a portion of the sample fluid 907 within a sample port of MPS device 102, e.g., examples in which the user is testing for de-clustering of MNPs after the addition of a biological sample. For example, as shown in FIG. 23, MNPs 2302 may be interlinked via one or more peptides 2310, and the addition of a biofluid including one or more target proteases 2320 to a fluid including MNPs 2302 may cleave peptides 2310, thereby reducing the hydrodynamic size of the interlinked MNPs 2302 and/or de-clustering MNPs 2302. The MPS device 102 may be initialized and/or calibrated with a signal corresponding to interlinked MNPs 2302 and may use either sample fluid 905 or sample fluid 907 to determine whether the added biofluid included one or more target proteases 2320 and/or an amount of one or more target proteases 2320. For example, bioassay system, e.g., diagnosis system 100, may include MPS device 102 including at least one conductive excitation coil configured to generate an alternating magnetic field including a first frequency and a second frequency, a sample mount configured to position a sample fluid 907 and/or 905 within the at least one conductive excitation coil, and a separator. The separator may be configured to separate a bound, interlinked, and/or clustered magnetic nanoparticle (MNP) 704 from an unbound, unlinked, and/or de-clustered MNP 2310, and the sample fluid 907 includes the unbound, unlinked, and/or de-clustered MNP 2310 and does not include the bound, interlinked, and/or clustered MNP 704. MPS device 102 includes at least one sensing conductive coil configured to sense a magnetic response of the sample 907 positioned within the sample mount to the alternating magnetic field. In some examples, a test material, e.g., including a surface functionalized MNP 2302 comprising a surface functionalization including a probe 2310 configured to bind to an analyte 2320 and/or another MNP 2302 and optionally a nonmagnetic microstructure configured to bind to the analyte 2320 and/or another MNP 2302, may be configured to de-cluster by the analyte 2320 (e.g., cleaving of peptides 2310 by target proteases 2320) upon combination with a biological sample including the analyte 2320.

FIGS. 10-14 illustrate example methods and devices of separating bound MNPs from unbound MNPs. Although described with reference to bound MNPs 704 and unbound MNPs 1602, 1604, the methods and devices of FIGS. 10-14 may be used to separate any of bound MNPs 702-710 from any of unbound MNPs 1502, 1602, 1604, 1702, 1802, 1902, 2002, 2004, 2102, 2202, 2204, 2302, 2404, 2504, 2530, or 2552.

FIG. 10 is a schematic diagram illustrating an example separator 1002 including a filter 1004, in accordance with one or more techniques of this disclosure. Separator 1002 may be an example of separator 154 of FIG. 1. In the example shown, separator 1002 includes a syringe 1006 including a plunger 1008 and filter 1004.

Syringe 1006 may include a plurality of bound MNPs 704 and unbound MNPs 1602, 1604, e.g., after a user has introduced a biological sample including analytes 1520 to a fluid comprising a plurality of MNPs 1602, 1604 that have been surface functionalized and include capture probes 1610, 1612, such as sample 903 described above. Syringe 1006 also includes filter 1004 configured to separate bound MNPs 704 from unbound MNPs 1602, 1604. Plunger 1008 may be configured to push fluid and unbound MNPs 1602, 1604 of sample 903 through filter 1004. After plunger 1008 is depressed pushing fluid through and unbound MNPs 1602, 1604, bound MNPs 704 may remain on or within filter 1004 and within syringe 1002, while unbound MNPs 1602, 1604 are pushed out of syringe 1002. In the example shown, after depressing plunger 1008, some fluid may remain, e.g., fluid sample 905, having a higher concentration of bound MNPs 704, e.g., relative to fluid sample 903. Syringe 1002, filter 1004, sample 905, and/or a sample including the higher concentration of bound MNPs 704 may then be used in a diagnosis system, e.g., diagnosis system 100 and MPS device 102.

In some examples, filter 1004 is configured to separate bound MNPs 704 from unbound MNPs 1602, 1604 by size, e.g., hydrodynamic size. Filter 1004 may have a pore size that is less than the hydrodynamic size of the bound MNP 704 and greater than a hydrodynamic size of the unbound MNP 1602, 1604. For example, filter 1004 may have a pore size of less than or equal to 1 micrometer, unbound MNPs 1602, 1604 may have a maximum dimension of less than 500 nanometers, and bound MNPs 704 may have a minimum dimension of at least 1 micrometer. In other examples, filter 1004 may have a pore size of less than or equal to 2 micrometers, less than or equal to 5 micrometers, or less than or equal to 10 micrometers. In some examples, filter 1004 may have a pore size that is about 2 times the hydrodynamic size of unbound MNPs (e.g., unbound MNPs 1602, 1604), or 3 times the hydrodynamic size of unbound MNPs, or 4 or more times the hydrodynamic size of unbound MNPs. In some examples, filter 1004 may have a diameter of at least 3 millimeters (mm), at least 5 mm, at least 7 mm, at least 10 mm, at least 15 mm, or at least 20 mm.

On other example, separator 1002 may include other apparatus instead of a syringe, e.g., a conduit through which a sample including bound MNPs 704 and unbound MNPs 1602, 1604 may flow (e.g., in a fluid) to encounter filter 1004. Separator 1002 may include a plunger, piston, pump, or any means for pushing the fluid including bound MNPs 704 and unbound MNPs 1602, 1604, e.g., via pressure of the fluid, through filter 1004 to separate bound MNPs, which may not pass through filter 1004, from unbound MNPs 1602, 1604, which may pass through filter 1004.

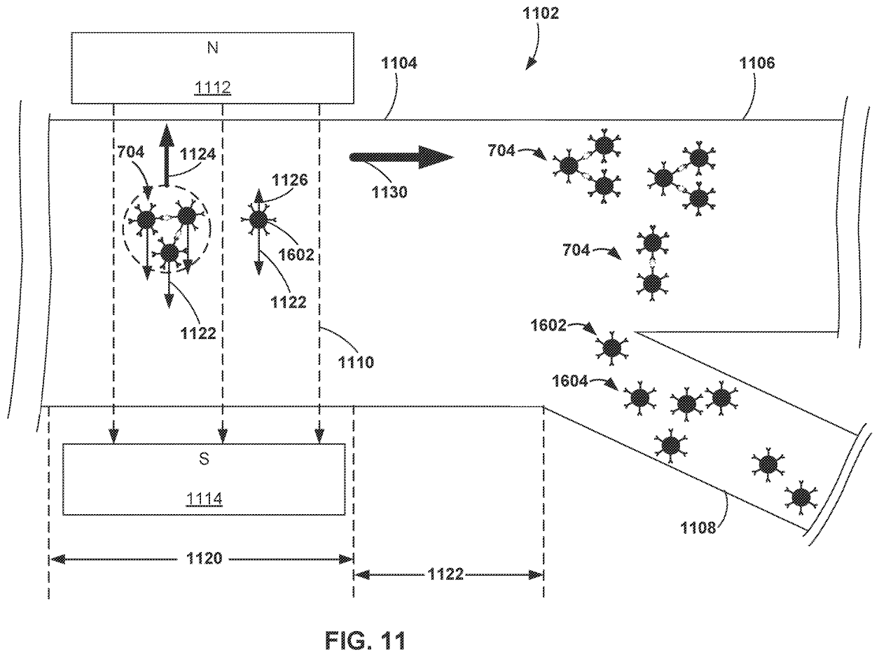

FIG. 11 is a schematic diagram illustrating an example magnetic separator 1102 including magnets 1112, 1114, in accordance with one or more techniques of this disclosure. Separator 1102 may be an example of separator 154 of FIG. 1. In the example shown, separator 1102 includes conduit 1104 including downstream branches 1106 and 1108.

Conduit 1104 may include a fluid flowing in direction 1130. The fluid may include a plurality of bound MNPs 704 and unbound MNPs 1602, 1604, e.g., after a user has introduced a biological sample including analytes 1520 to the fluid comprising a plurality of MNPs 1602, 1604 that have been surface functionalized and include capture probes 1610, 1612, such as sample 903 described above.

Magnets 1112, 1114 may be arranged to cause a magnetic field 1110 within a portion of conduit 1104 upstream from branches 1106, 1108. The magnetic field 1110 may have a gradient, e.g., between the north (N) magnet 1112 and the south(S) magnet 1114 and may also be referred to as gradient magnetic field 1110. Gradient magnetic field 1110 causes a magnetic force 1122 on bound MNPs 704 and unbound MNPs 1602, 1604 in a direction different from that of flow direction 1130, e.g., substantially perpendicular to flow direction 1130. The magnetic force 1122 (e.g., a magnetic attraction force) on the MNPs of the bound MNPs 704 and unbound MNPs 1602, 1604 may be proportional to the magnetic field gradient and the absolute field strength acting on the MNPs. Magnets 112, 1114 may be permanent magnets, electromagnets, or any suitable magnets. Although two magnets are shown, magnetic separator 1102 may include more or fewer magnets, e.g., one magnet, or three or more magnets. Although shown as magnets on opposite sides of channel/conduit 1104, magnetic separator 1102 may include one or more magnets in any suitable configuration to cause a gradient magnetic field 1110 within a portion of channel/conduit 1104.

In the example shown, the MNPs may be substantially the same (e.g., same size, material, magnetization) and magnets 1112, 1114 cause a uniform magnetic field within the portion of conduit 1104, and magnetic force 1122 is the same on each of the MNPs of bound MNPs 704 and unbound MNPs 1602, 1604. As the bound MNPs 704 and unbound MNPs 1602, 1604 accelerate and move within the fluid in the direction of magnetic force 1122, bound MNPs 704 may have a fluid resistance 1124 to motion (e.g., fluid friction) that is larger than a fluid resistance 1126 of the unbound MNPs 1602, 1604, e.g., due to the larger hydrodynamic size of bound MNPs 704. Fluid resistances 1124, 1126 are in the opposite direction from magnetic force 1122, and bound MNPs 704 will accelerate less and move slower in the direction of magnetic force 1122 than unbound MNPs 1602, 1604 in within the fluid, thereby spatially separating within the fluid and conduit 1104. Bound MNPs 704 may then flow into downstream branch 1106, and unbound MNPs 1602, 1604 may flow into downstream branch 1108. In some examples, the fluid may have a density and flow rate configured to separate bound MNPs 704 from unbound MNPs 1602, 1604, e.g., in conjunction with a length 1120 of conduit 1104 including the gradient magnetic field 1110 and the length 1122 of conduit from gradient magnetic field 1110 to branches 1106, 1108.

In some examples, conduit 1104 and branches 1106, 1108 may comprise channels and/or microfluidic channels. For example, a user may add a biological sample including analytes 1520 to a container including a fluid comprising a plurality of MNPs 1602, 1604, e.g., container 902. Channel 1104 may be in fluid communication with container 902, and branches 1106 and 1108 may be first and second outlets. First branch/outlet 1106 may be positioned proximate to a first side of the cross-sectional area of the channel 1104 (e.g., a side corresponding to magnet 1112 in the example shown) and second branch/outlet 1108 may be positioned opposite first branch/outlet 1108, e.g., proximate to a second side of the cross-sectional area of the channel 1104 corresponding to magnet 1114 in the example shown. One or more magnets 1112, 1114 may be configured to apply the gradient magnetic field 1110 to a portion of the length, e.g., length 1120, of channel 1104 upstream from the first and second outlets 1106, 1108. Gradient magnetic field 1110 may be configured to cause a magnetic force 1122 on bound MNP 704 and the unbound MNPs 1602, 1604 in a direction substantially perpendicular to the direction of a flow 1130 of the sample fluid, wherein a fluid resistance of the sample fluid is proportional to hydrodynamic size causing the unbound MNPs 1602, 1604 to move in the direction of the magnetic force 1122 by a greater amount than the bound MNP 704, e.g., such that bound MNPs 704 flow with the fluid through first outlet 1106 and unbound MNPs 1602, 1604 flow with the fluid through second outlet 1108.

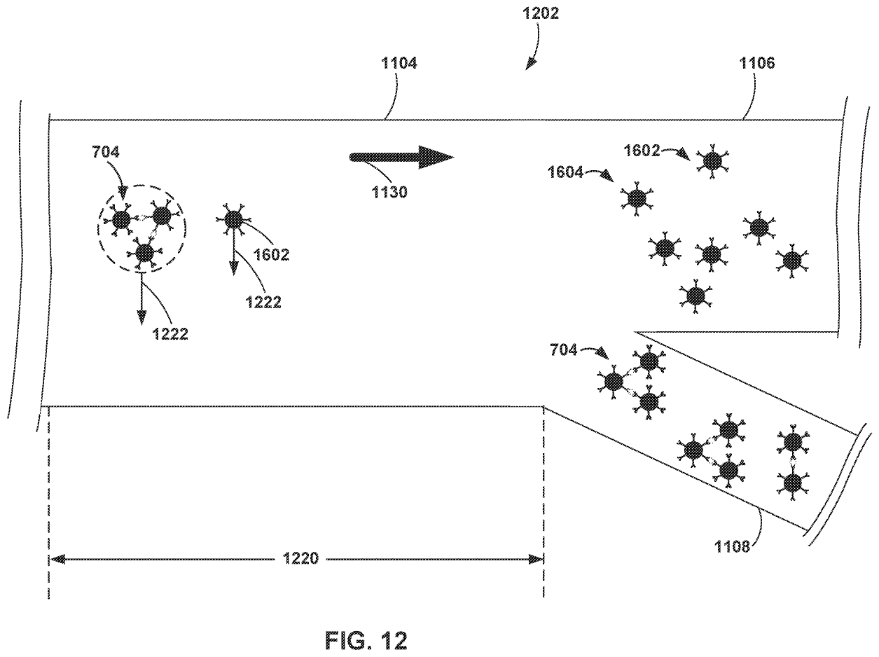

FIG. 12 is a schematic diagram illustrating an example gravitational separator 1202, in accordance with one or more techniques of this disclosure. Separator 1202 may be an example of separator 154 of FIG. 1. In the example shown, separator 1202 includes conduit 1104 including downstream branches 1106 and 1108, which may be substantially similar to conduit/channel 1104 and downstream branches/outlets 1106, 1108 described above.

Similar to separator 1102, conduit 1104 of separator 1202 may include a fluid flowing in direction 1130. The fluid may include a plurality of bound MNPs 704 and unbound MNPs 1602, 1604, e.g., after a user has introduced a biological sample including analytes 1520 to the fluid comprising a plurality of MNPs 1602, 1604 that have been surface functionalized and include capture probes 1610, 1612, such as sample 903 described above.

Conduit 1104 may be arranged such that gravity 1222 causes a force on bound MNPs 704 and unbound MNPs 1602, 1604 in a direction different from that of flow direction 1130, e.g., substantially perpendicular to flow direction 1130. The heavier bound MNPs 704 having a larger hydrodynamic size, e.g., relative to unbound MNPs 1602, 1604, may sediment (e.g., settle) towards the direction of gravity 1222, e.g., towards the “bottom” of conduit/channel 1104. Bound MNPs 704 may then flow into downstream branch 1108, and unbound MNPs 1602, 1604 may flow into downstream branch 1106. In some examples, the fluid may have a density and flow rate configured to separate bound MNPs 704 from unbound MNPs 1602, 1604, e.g., in conjunction with a length 1220 of a portion of channel/conduit 1104 to outlets/branches 1106, 1108.

In some examples, conduit 1104 and branches 1106, 1108 may comprise channels and/or microfluidic channels. For example, a user may add a biological sample including analytes 1520 to a container including a fluid comprising a plurality of MNPs 1602, 1604, e.g., container 902. Channel 1104 may be in fluid communication with container 902, and branches 1106 and 1108 may be first and second outlets. First branch/outlet 1106 may be positioned proximate to a first side of the cross-sectional area of the channel 1104 and second branch/outlet 1108 may be positioned opposite first branch/outlet 1108, e.g., proximate to a second side of the cross-sectional area of the channel 1104 opposite the first side of the cross-sectional area of channel 104. Conduit/channel 1104 and outlets/branches 14106, 1108 may be arranged (e.g., horizontal relative to gravity) such that gravity 1222 causes bound MNPs 704 to move, e.g., sediment, more than unbound MNPs 1602, 1604 in a direction substantially perpendicular to the direction of a flow 1130 of the sample fluid, e.g., such that 704 unbound MNPs 1602, 1604 flow with the fluid through first outlet 1106 and bound MNPs flow with the fluid through second outlet 1108.

FIG. 13A is a schematic diagram illustrating an example centrifuge separator 1302, in accordance with one or more techniques of this disclosure. FIG. 13B is a schematic diagram illustrating an example container including separated bound MNPs 704 and unbound MNPs 1602, 1604, e.g., after having been separated by separator 1302, in accordance with one or more techniques of this disclosure. Separator 1302 may be an example of separator 154 of FIG. 1. In the example shown, separator 1302 includes container 902 and a centrifuge 1302.

In the example shown, container 902 may include a fluid including a plurality of bound MNPs 704 and unbound MNPs 1602, 1604, e.g., after a user has introduced a biological sample including analytes 1520 to the fluid comprising a plurality of MNPs 1602, 1604 that have been surface functionalized and include capture probes 1610, 1612, such as sample 903 described above. Container 902 is configured to be positioned in centrifuge 1304, and centrifuge 1304 is configured to spin and/or centrifuge sample 903. For example, centrifuge 1304 is configured to centrifuge sample 903 to cause bound MNPs 704 to sediment to a bottom portion of the sample fluid, e.g., sample fluid 905 at a bottom portion of container 902, while bound MNPs 1602, 1604 remain in a supernatant 909 portion of the sample fluid. The supernatant 909 may be removed, and the remaining sample fluid 905, having a higher concentration of bound MNPs 704, may be used with MPS device 102, or the process repeated to further increase the concentration of bound MNPs 704 in sample fluid 905.

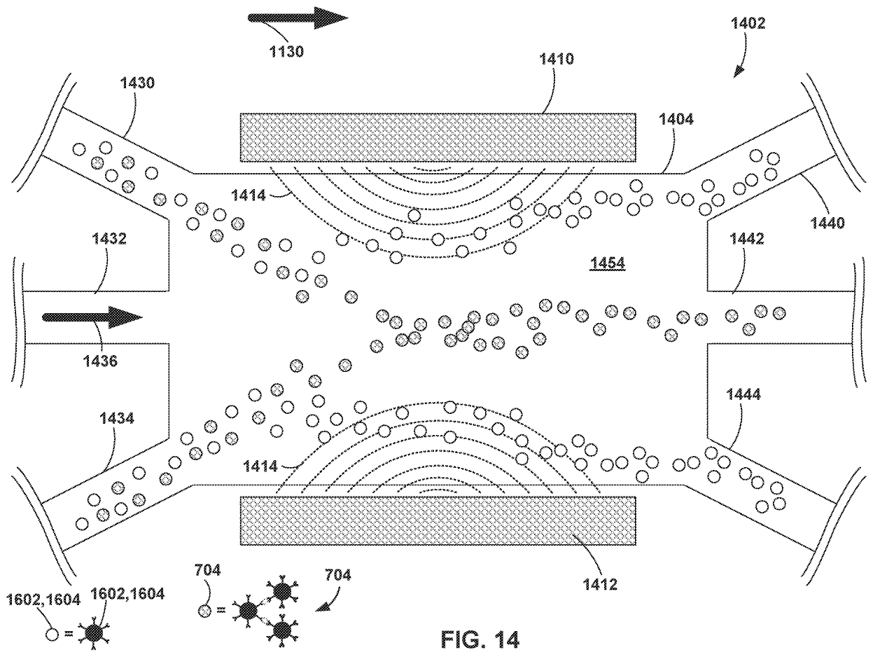

FIG. 14 is a schematic diagram illustrating an example separator 1402, in accordance with one or more techniques of this disclosure. Separator 1402 may be an example of separator 154 of FIG. 1. In the example shown, separator 1402 includes conduit/channel 1402 and interdigital transducers (IDTs) 1410, 1412. Separator 1402 may be a surface acoustic wave (SAW) separator configured to separate bound MNPs 704 from unbound MNPs 1602, 1604 by hydrodynamic size via acoustic waves 1414, which may be surface acoustic waves (SAWs) 1414 and/or standing acoustic waves.

In the example shown, conduit/channel 1402 may include inlets 1430, 1432, 1434 and outlets 1440, 1442, 1444. Inlet 1434 includes sheath flow 1436 of a fluid in the direction of flow 1130 of sample fluid 903. Sample fluid 903, including bound MNPs 704 and unbound MNPs 1602, 1604, flows from inlets 1430, 1434, into separation region 1454. IDTs 1410, 1412 cause SAWs 1414 within separation region 1454, causing separation of bound MNPs 704 from unbound MNPs 1602, 1604. For example, sheath fluid 1436 flows from inlet 1432 and joins flows of sample fluid 903 from inlets 1430, 1434 in separation region 1454, and IDTs 1410, 1412 cause SAWs 1414 within the flow of the joined sample fluid 903 and sheath fluid 1436 within separation region 1454. The SAWs 1414 then cause the bound MNPs 704 to move, e.g., in a direction different from flow direction 1130, which may be substantially perpendicular to flow direction 1130, into the sheath fluid 1436 flow. For example, bound MNPs 704 may have a hydrodynamic size large enough such that SAWs 1414 induce motion in a direction towards the radial center of separation region 1454 that is great enough to overcome a fluid resistance to motion, e.g., from sheath fluid 1436. Unbound MNPs 1602, 1604 may have a relatively smaller hydrodynamic size such that SAWs 1414 do not induce motion in a direction towards the radial center of separation region 1454 that is great enough to overcome a fluid resistance to motion, e.g., from sheath fluid 1436, and unbound MNPs 1602, 1604 may flow in a radially outwards portion of separation region 1454. Bound MNPs 704 may then become entrained within, and/or flow within, sheath fluid 1436 out of separation region 1454 through outlet 1442 (e.g., at a radially central portion of channel/conduit 1404). Unbound MNPs 1602, 1604 may then be constrained from flowing within sheath fluid 1436, and may flow along the radially outwards portions of separation region 1454 and through outlets 1440, 1444 (e.g., at radially outwards portions of channel/conduit 1404). SAWs 1414 may be configured to cause bound MNPs 704, having a hydrodynamic size greater than a threshold hydrodynamic size, to move within the joined sheath fluid 1436 and sample fluid 903, in a non-flow direction, e.g., to become entrained in sheath flow 1436. SAWs 1414 may be configured to cause unbound MNPs 1602, 1604, having a hydrodynamic size less than the threshold hydrodynamic size, to not move within the joined sheath fluid 1436 and sample fluid 903, in a non-flow direction, and sheath fluid 1436 may prevent unbound MNPs 1602, 1604 from becoming entrained in sheath flow 1436. Separator 1402 is configured to keep the flow of the sheath fluid 1436 separate from the flow of the sample fluid 903 downstream from the SAWs 1414, thus separating bound MNPs 704 from unbound MNPs 1602, 1604.

FIGS. 15-25C illustrate example chemical and mechanical processes that may cause MNP hydrodynamic size to change in the presence of one or more analytes, and may be detectable via MPS, for example, by using MPS handheld device 102. In some examples, the example chemical and mechanical processes causing MNP hydrodynamic size change may be distinguishable via harmonic amplitude and harmonic amplitude ratio changes.

FIG. 15 is an illustration of an example surface functionalized MNP 1502, in accordance with one or more techniques of this disclosure. In the example shown, MNP 1502 may be surface functionalized via a coating of one or more monoclonal antibodies 1510. Monoclonal antibodies 1510 may be selected to bind to a single antigen or antibody. In some examples, one or more antigens 1520 may bind to the one or more monoclonal antibodies 1510, for example, via addition of a biofluid containing antigens 1520 to sample vial 108 including MNPs 1502. In some examples, antigens 1520 may be one or more analytes, for example, antigens 1520 may be one or more analytes of a coronavirus such as human coronavirus 229E, human coronavirus OC43, SARS-COV (2003), HCoV NL63 (2004), HKU1 (2005), MERS-COV (2102), SARS-COV-2, and the like. In some examples, antigens 1520 may be any of a membrane, an envelope, a structure protein, a Hemagglutinin esterase protean, a nucleocapsid protein inside the envelop, and the like, of a coronavirus. MNP 1502 may have an increased hydrodynamic size as a result of the binding of antigens 1520 to monoclonal antibodies 1510. The increased hydrodynamic size may change the relaxation time of MNP 1502 in response to the applied magnetic fields described herein, which may affect the magnetic fields detected by the pick-up coil. MNP 1502, when bound to antigen 1520, may be an example of a bound MNP 702 (FIG. 7A) with an increased hydrodynamic size without forming a cluster (e.g., without clustering with one or more other MNP).

FIG. 16 is an illustration of example surface functionalized MNPs 1602 and 1604, in accordance with one or more techniques of this disclosure. In the example shown, MNP 1602 may be surface functionalized via a coating of one or more antibodies 1610, and MNP 1604 may be surface functionalized via a coating of one or more antibodies 1612, where antibodies 1610 may be different from antibodies 1612. For example, antibodies 1610 may be configured to bind to a first portion of an antigen 1520 and antibodies 1612 may be configured to bind to a second portion of the antigen 1520. In some examples, one or more antigens 1520 may bind to either of the one or more antibodies 1610 and 1612, for example, via addition of a biofluid containing antigens 1520 to sample vial 108 including MNPs 1602 and 1604. In the example shown, MNPs 1602 and 1604 may cluster as a result of binding with antigens 1520, and in some examples, MNPs 1602 and 1604 may have an increased hydrodynamic size as a result of the binding of antigens 1520 to antibodies 1610 and 1612. The configuration of FIG. 16 may be more sensitive than the configuration of FIG. 15, as the hydrodynamic size of particles changes to a greater extent in response to binding of an antigen 1520 to antibodies 1610 and 1612. In some examples, antigen 1520 may be substantially similar to antigen 1520 illustrated and described above with respect to FIG. 15. MNPs 1602, 1604 when bound to antigen 1520, may be an example of a bound MNP 704 (FIG. 7B) with an increased hydrodynamic size and with forming a cluster (e.g., with clustering with one or more other MNP).

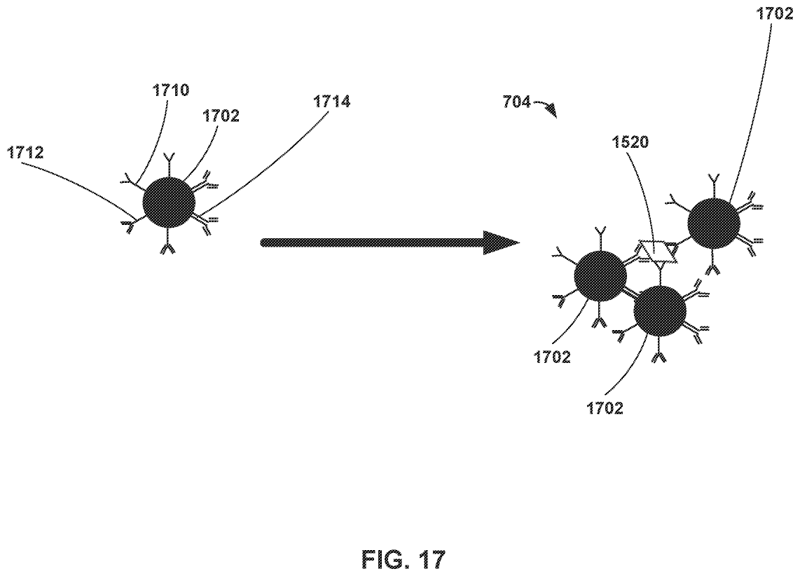

FIG. 17 is an illustration of an example surface functionalized MNP 1702, in accordance with one or more techniques of this disclosure. In the example shown, MNP 1702 may be surface functionalized via a coating of one or more polyclonal antibodies 1710, 1712, and 1714, where polyclonal antibodies 1710, 1712, and 1714 may be different from each other. In some examples, MNP 1702 may be coated with fewer or more than three polyclonal antibodies as illustrated in FIG. 17. In some examples, one or more antigens 1520 may bind to any of the one or more polyclonal antibodies 1710, 1712, and 1714, for example, via addition of a biofluid containing antigens 1520 to sample vial 108 including MNPs 1702. For example, each of polyclonal antibodies 1710, 1712, and 1714 may be configured to bind to different binding sites of antigen 1520. In the example shown, a plurality of MNPs 1702 may cluster as a result of binding with antigen(s) 1520, and in some examples, MNPs 1702 may have an increased hydrodynamic size as a result of the binding of antigens 1520 to antibodies 1710, 1712, and/or 1714. In some examples, antigen 1520 may be substantially similar to antigen 1520 illustrated and described above with respect to FIG. 15. MNPs 1702 when bound to antigen 1520, may be an example of a bound MNP 704 (FIG. 7B) with an increased hydrodynamic size and with forming a cluster (e.g., with clustering with one or more other MNP).

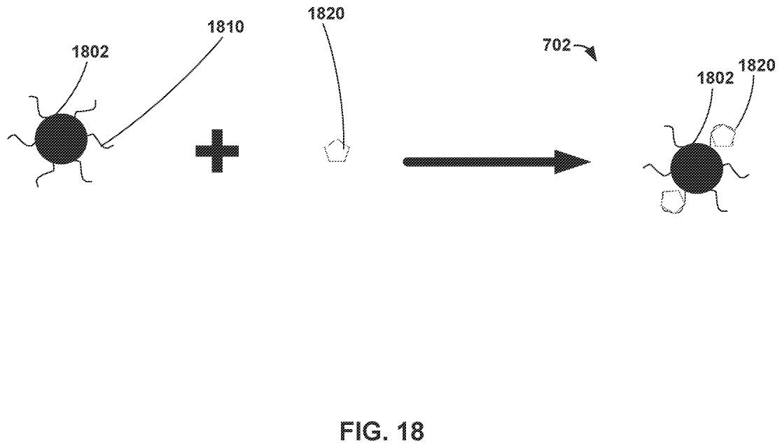

FIG. 18 is an illustration of an example surface functionalized MNP 1802, in accordance with one or more techniques of this disclosure. In the example shown, MNP 1802 may be surface functionalized via a coating of one or more single strand DNA and/or RNA 1810. In some examples, one or more DNA-binding proteins 1820 may interact and/or bind to the one or more single strand DNA and/or RNA 1810, for example, via addition of a biofluid containing DNA-binding proteins 1820 to sample vial 108 including MNPs 1802. In some examples, MNP 1802 may have an increased hydrodynamic size as a result of the interaction/binding of DNA-binding proteins 1820 with single strand DNA and/or RNA 1810. In some examples, DNA-binding proteins 1820 may be one or more analytes, for example, DNA-binding proteins 1820 may be one or more analytes of a coronavirus such as human coronavirus 229E, human coronavirus OC43, SARS-COV (2003), HCoV NL63 (2004), HKU1 (2005), MERS-COV (2102), SARS-COV-2, and the like. In some examples, DNA-binding proteins 1820 may be any of a membrane, an envelope, a structure protein, a Hemagglutinin esterase protean, a nucleocapsid protein inside the envelop, and the like, of a coronavirus. In some examples, DNA-binding proteins 1820 may include transcription factors which modulate the process of transcription, various polymerases, nucleases which cleave DNA molecules, and histones involved in chromosome packaging and transcription in the cell nucleus. MNP 1802, when bound to DNA-binding proteins 1820, may be an example of a bound MNP 702 (FIG. 7A) with an increased hydrodynamic size without forming a cluster (e.g., without clustering with one or more other MNP).

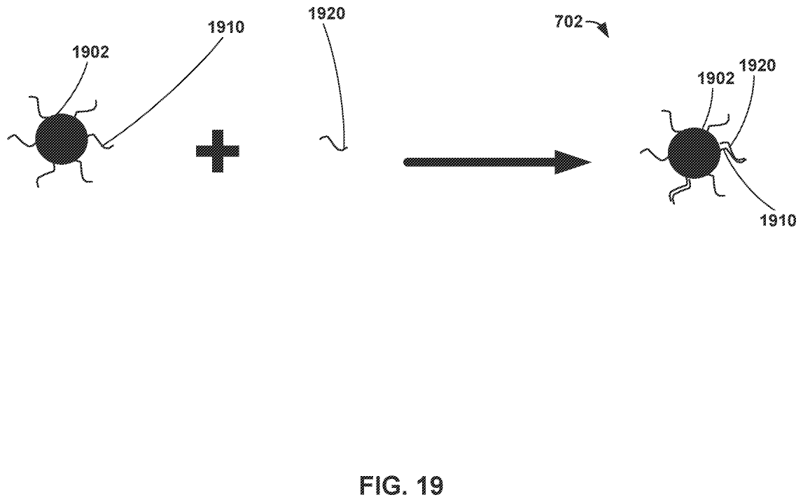

FIG. 19 is an illustration of an example surface functionalized MNP 1902, in accordance with one or more techniques of this disclosure. In the example shown, MNP 1902 may be surface functionalized via a coating of one or more reverse complementary single strand DNA and/or RNA 1910. In some examples, one or more single strand DNA and/or RNA 1920 may interact and/or bind to the one or more reverse complementary single strand DNA and/or RNA 1910, for example, via addition of a biofluid containing single strand DNA and/or RNA 1920 to sample vial 108 including MNPs 1902. In some examples, MNPs 1902 may have an increased hydrodynamic size as a result of the interaction/binding of single strand DNA and/or RNA 1920 with reverse complementary single strand DNA and/or RNA 1910. In some examples, single strand DNA and/or RNA 1920 may be one or more analytes, for example, single strand DNA and/or RNA 1920 may be one or more analytes of a coronavirus such as human coronavirus 229E, human coronavirus OC43, SARS-COV (2003), HCoV NL63 (2004), HKU1 (2005), MERS-COV (2102), SARS-COV-2, and the like. In some examples, single strand DNA and/or RNA 1920 may be any of a single stranded DNA (ss-DNA) and/or RNA (ss-RNA), and the like, of a coronavirus. MNP 1902, when bound to single strand DNA and/or RNA 1920, may be an example of a bound MNP 702 (FIG. 7A) with an increased hydrodynamic size without forming a cluster (e.g., without clustering with one or more other MNP).

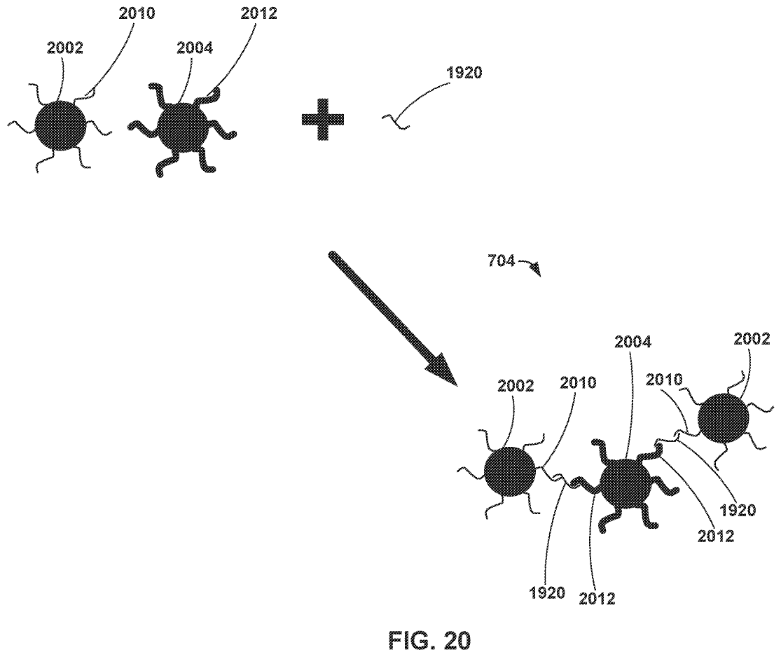

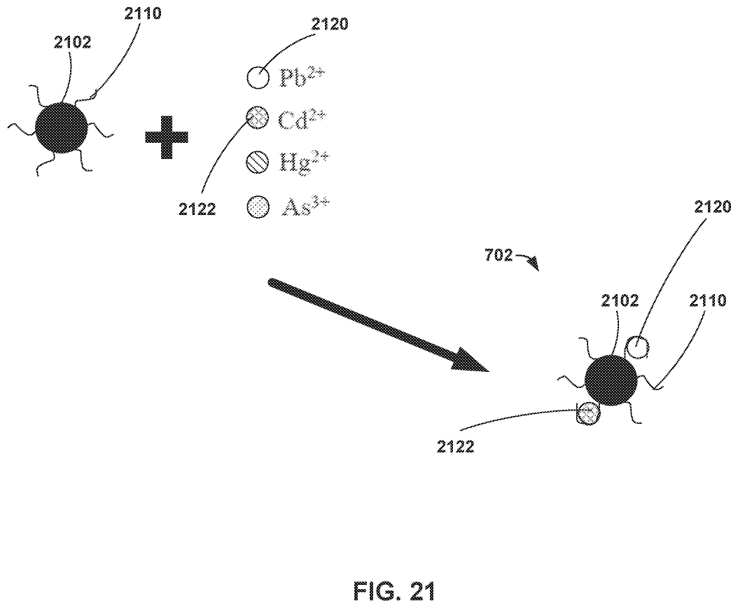

FIG. 20 is an illustration of example surface functionalized MNPs 2002 and 2004, in accordance with one or more techniques of this disclosure. In the example shown, MNPs 2002 and 2004 may be surface functionalized via a coating of one or more single strand DNA and/or RNA 2010 and 2012, respectively. In some examples, single strand DNA and/or RNA 2510 and 2012 may be different from each other, but still at least partially pair with different portions of one or more single strand DNA and/or RNA 1920. In some examples, one or more single strand DNA and/or RNA 1920 may interact, bind, and/or partially pair with the one or more single strand DNA and/or RNA 2510 and 2012, for example, via addition of a biofluid containing single strand DNA and/or RNA 1920 to sample vial 108 including MNPs 2002 and 2004. In the example shown, MNPs 2002 and 2004 may cluster as a result of the interaction/binding/at least partial pairing with single strand DNA and/or RNA 1920, and in some examples, MNPs 2002 and 2004 may have an increased hydrodynamic size as a result of the interaction/binding/at least partial pairing of single strand DNA and/or RNA 1920 with single strand DNA and/or RNA 2510 and 2012. In some examples, single strand DNA and/or RNA 1920 may be one or more analytes, for example, single strand DNA and/or RNA 1920 may be substantially similar to single strand DNA and/or RNA 1920 illustrated and described above with respect to FIG. 19. MNPs 2002, 2004 when bound to single strand DNA and/or RNA 1920, may be an example of a bound MNP 704 (FIG. 7B) with an increased hydrodynamic size and with forming a cluster (e.g., with clustering with one or more other MNP).

FIG. 21 is an illustration of an example surface functionalized MNP 2102, in accordance with one or more techniques of this disclosure. In the example shown, MNP 2102 may be surface functionalized via a coating of one or more differing types of structure switching strand DNA and/or RNA 2110. In some examples, one or more heavy metal ions 2120 and/or 2122 may interact and/or bind to the one or more differing types of structure switching strand DNA and/or RNA 2110, for example, via addition of a biofluid, drinking water, or any fluid containing heavy metal ions 2120 and/or 2122 to sample vial 108 including MNPs 2102. In some examples, MNPs 2102 may have an increased or decreased hydrodynamic size as a result of the interaction/binding of heavy metal ions 2120 and/or 2122 with the one or more differing types of structure switching strand DNA and/or RNA 2110. MNP 2102, when bound to heavy metal ions 2120 and/or 2122, may be an example of a bound MNP 702 (FIG. 7A) with an increased hydrodynamic size without forming a cluster (e.g., without clustering with one or more other MNP).

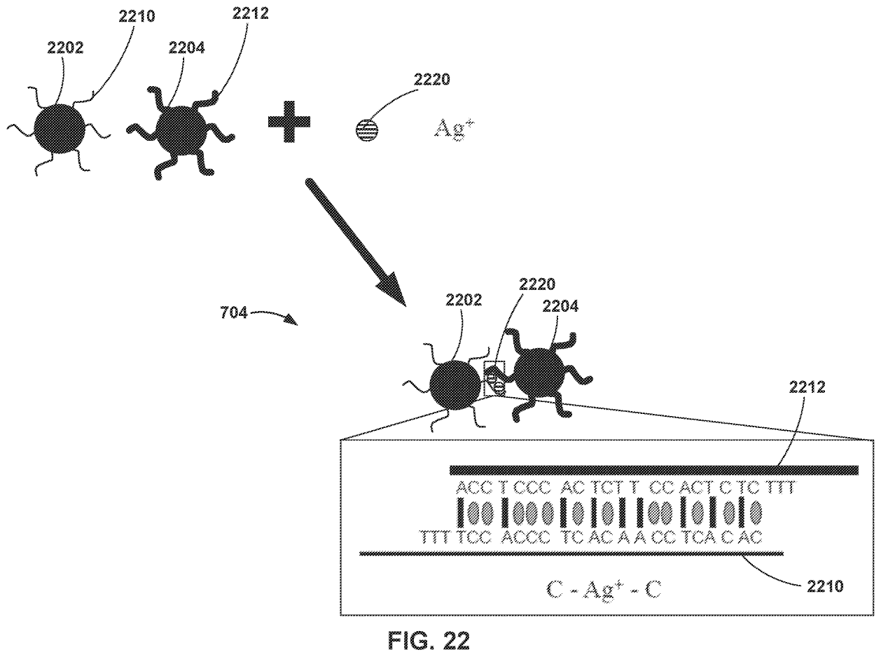

FIG. 22 is an illustration of example surface functionalized MNPs 2202 and 2204, in accordance with one or more techniques of this disclosure. In the example shown, MNPs 2202 and 2204 may be surface functionalized via a coating of one or more single strand DNA and/or RNA 2210 and 2212, respectively. In some examples, single strand DNA and/or RNA 2210 and 2212 may be different from each other may include partially mismatched single strand RNA and/or DNA. In some examples, one or more heavy metal ions 2220 may interact, bind, and/or at least partially match with the one or more single strand DNA and/or RNA 2010 and 2012, for example, via addition of a biofluid, drinking water, etc., containing one or more heavy metal ions 2220 to sample vial 108 including MNPs 2202 and 2204. In the example shown, MNPs 2202 and 2204 may cluster as a result of the interaction/binding/at least partial matching with heavy metal ions 2220, and in some examples, MNPs 20202 and 2204 may have an increased hydrodynamic size as a result of the interaction/binding/at least partial matching of heavy metal ions 2220 with single strand DNA and/or RNA 2210 and 2212. MNPs 2202, 2204 when bound to heavy metal ions 2220, may be an example of a bound MNP 704 (FIG. 7B) with an increased hydrodynamic size and with forming a cluster (e.g., with clustering with one or more other MNP).

FIG. 23 is an illustration of an example surface functionalized MNPs 2302, in accordance with one or more techniques of this disclosure. In the example shown, a plurality of MNPs 2302 may be interlinked via one or more peptides 2310. In some examples, one or more target proteases 2320 may interact with and/or cleave peptides 2310, for example, via addition of a biofluid to sample vial 108 including MNPs 2302, thereby reducing the hydrodynamic size of the interlinked MNPs 2302. The presence of the target protease 2320 may result in a decreased hydrodynamic size of clusters of NMPs 2302 due to cleaving of peptides. For example, proteases 2320 may regulate multiple biological processes including cell differentiation, proteasomal degradation, inflammation, tissue remodeling, apoptosis, cell homeostasis, and coagulation. Consequently, the deregulation of proteolytic activity accounts for pathogenesis and progression of many diseases such as cardiovascular disease, inflammatory conditions, neurodegenerative disorders and cancer. MNPs 2302 when bound and/or interlinked via one or more peptides 2310, may be an example of a bound MNP 704 (FIG. 7B) with an increased hydrodynamic size and with forming a cluster (e.g., with clustering with one or more other MNP), and when one or more target proteases 2320 interact with and/or cleave peptides 2310, the resulting de-clustered and/or unlinked MNPs 2302 may be examples of unbound MNPs.

FIG. 24 is an illustration of example surface functionalized microbead 2402 and MNP 2404, in accordance with one or more techniques of this disclosure. In the example shown, nonmagnetic microbead 2402 may be surface functionalized via a coating of one or more antibodies 2410, and MNP 2404 may be surface functionalized via a coating of one or more antibodies 2412, where antibodies 2410 may be different from antibodies 2412. In some examples, microbead 2402 may be made of a nonmagnetic material, e.g., a polymer, polystyrene, or any suitable nonmagnetic material. In some examples, one or more antigens 1520 may bind to either of the one or more antibodies 2410 and 2412, for example, via addition of a biofluid containing antigens 1520 to sample vial 108 including MNPs 2402 and 2404. In the example shown, microbead 2402 and MNP 2404 may cluster as a result of antibodies 2410 and 2412 binding with antigens 1520, and in some examples, microbead 2402 and MNP 2404 may have an increased hydrodynamic size as a result of the binding of antigens 1520 to antibodies 2410 and 2412. In some examples, antigens 1520 may be one or more analytes, for example, antigens 1520 may be one or more analytes of a coronavirus such as human coronavirus 229E, human coronavirus OC43, SARS-COV (2003), HCoV NL63 (2004), HKU1 (2005), MERS-COV (2102), SARS-COV-2, and the like. In some examples, antigens 1520 may be any of a membrane, an envelope, a structure protein, a Hemagglutinin esterase protean, a nucleocapsid protein inside the envelop, and the like, of a coronavirus. In some examples, microbead 2402 may replace one of the types of MNP 1602 and 1604 in the example shown in FIG. 16 above. In some examples, antigen 1520 may be substantially similar to antigen 1520 illustrated and described above with respect to FIG. 15. MNP 2404 when bound to antigens 1520 and microbead 2402, may be an example of a bound MNP 706 (FIG. 7C) with an increased hydrodynamic size and with forming a cluster (e.g., with clustering with one or more other MNP and/or one or more other microbead).

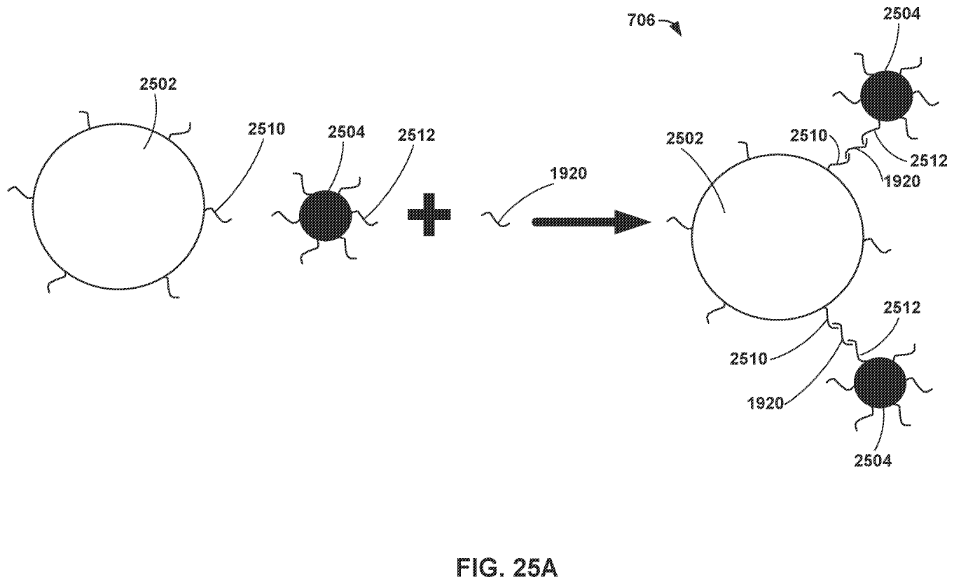

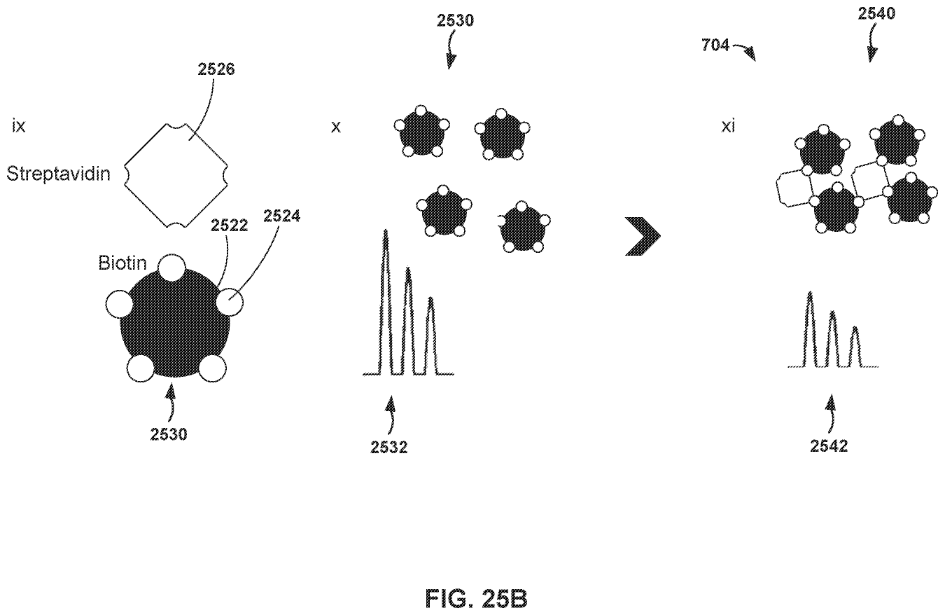

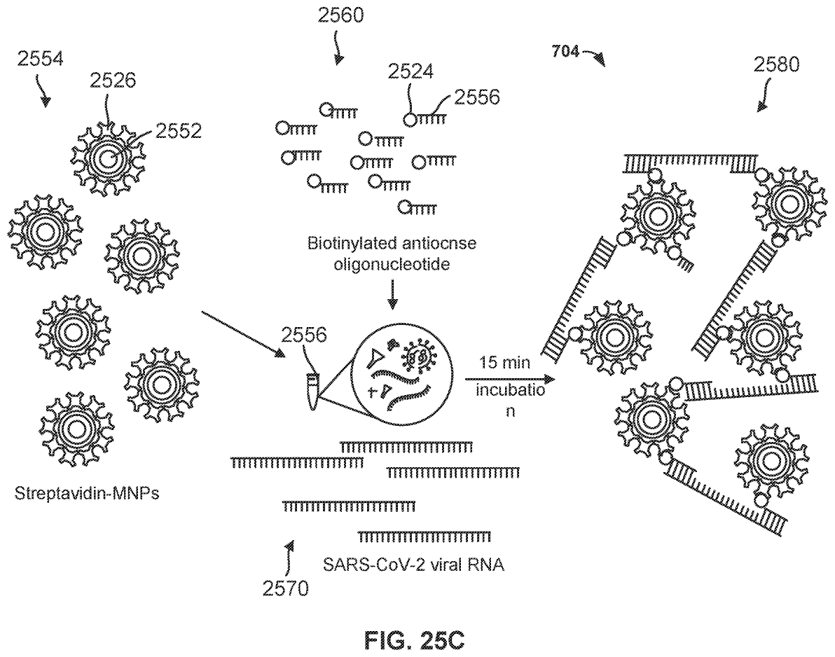

FIG. 25A is an illustration of example surface functionalized nonmagnetic microbead 2502 and MNP 2504, in accordance with one or more techniques of this disclosure. In the example shown, microbead 2502 and MNP 2504 may be surface functionalized via a coating of one or more single strand DNA and/or RNA 2510 and 2512, respectively. In some examples, single strand DNA and/or RNA 2510 and 2512 may be different from each other, but still at least partially pair with one or more single strand DNA and/or RNA 1920. In some examples, one or more single strand DNA and/or RNA 1920 may interact, bind, and/or partially pair with the one or more single strand DNA and/or RNA 2510 and 2512, for example, via addition of a biofluid containing single strand DNA and/or RNA 1920 to sample vial 108 including microbeads 2502 and MNPs 2504. In the example shown, microbeads 2502 and MNPs 2504 may cluster as a result of the interaction/binding/at least partial pairing with single strand DNA and/or RNA 1920, and in some examples, microbeads 2502 and MNPs 2504 may have an increased hydrodynamic size as a result of the interaction/binding/at least partial pairing of single strand DNA and/or RNA 1920 with single strand DNA and/or RNA 2510 and 2512. In some examples, single strand DNA and/or RNA 1920 may be one or more analytes, for example, single strand DNA and/or RNA 1920 may be substantially similar to single strand DNA and/or RNA 1920 illustrated and described above with respect to FIG. 19. In some examples, microbead 2502 may replace one of the types of MNP 2002 and 2004 in the example shown in FIG. 20 above.

Additionally, in some examples, microbeads 2402, 2502 may replace one of the types of MNP 2202, 2204 as described and illustrated above with respect to FIG. 22, and/or replace some of or be added to MNPs 2302 as described and illustrated above with respect to FIG. 23. Replacement of one of the types of MNP 1502, 1602, 2002, 2004, 2202, 2204, and 2302 by microbeads 2402 and/or 2502 in the examples described above with respect to FIGS. 16, 20, 22-25, respectively, may increase hydrodynamic size, and thereby may increase the sensitivity of MPS measurements. MNP 2504 when bound to single strand DNA and/or RNA 1920 and microbead 2502, may be an example of a bound MNP 706 (FIG. 7C) with an increased hydrodynamic size and with forming a cluster (e.g., with clustering with one or more other MNP and/or one or more other microbead).

FIG. 25B is an illustration of example surface functionalized MNP 2522, in accordance with one or more techniques of this disclosure. In the example shown, MNP 2522 may be surface functionalized via a coating of biotin 2524. Biotin 2524 may be selected to bind to one or more streptavidin protein 2526.

Streptavidin is a homo-tetramer with an extraordinarily high affinity for biotin, e.g., 1 mol of streptavidin can bind with 4 mol of biotin. In the example shown, well-dispersed biotinylated MNPs 2530 show high dynamic magnetic responses to external oscillating fields as well as large harmonic amplitudes 2532. In the presence of streptavidin 2526, biotinylated MNPs 2530 may cross-link and form clusters 2540 on streptavidin homo-tetramers. The clustering of MNPs may weaken the dynamic magnetic responses, and as a result, the harmonic amplitudes drop and/or reduce relative to well-dispersed MNPs, e.g., illustrated as harmonic amplitudes 2542 reduced relative to harmonic amplitudes 2532 in the example shown. The difference in harmonic amplitude reduction may be used to quantitatively analyze the amount and concentration of streptavidin in a sample. An MPS-based bioassay, e.g., such as with MPS handheld device 102, may not require removing unbound target analytes (e.g., streptavidin 2526 in the example shown) and may be a wash-free, one-step test that is accessible by a layperson in nonclinical settings. MNP 2522 when bound to streptavidin 2526, may be an example of a bound MNP 704 (FIG. 7B) with an increased hydrodynamic size and with forming a cluster (e.g., with clustering with one or more other MNPs, e.g., forming one or more cluster 2540).

FIG. 25C is an illustration of example surface functionalized MNPs 2552, in accordance with one or more techniques of this disclosure. In the example shown, MNP 2522 may be surface functionalized via a coating of biotin 2524 and streptavidin proteins 2526, e.g., which may form streptavidin-saturated biotinylated MNPs 2554. For example, a significant portion of biotin 2524 of each MNP 2552 may bind with streptavidin proteins 2526 individually, e.g., such that each MNP 2554 does not form clusters such as clusters 2540 of FIG. 25B, but may remain dispersed because a threshold amount of the biotin, or substantially all of the biotin, of the surface functionalized MNPs 2554 may already be bound to streptavidin proteins 2526.

In some examples, one or more antisense oligonucleotides 2556 may be bound to biotin 2524 to form biotinylated antisense oligonucleotides 2560 and introduced to streptavidin-saturated biotinylated MNPs 2554, e.g., alone or in combination with a biological sample including analytes 2570 of a coronavirus such as human coronavirus 229E, human coronavirus OC43, SARS-COV (2003), HCOV NL63 (2004), HKU1 (2005), MERS-CoV (2102), SARS-COV-2, and the like. For example, analyte 2570 may be a SARS-COV-2 viral RNA. In some examples, streptavidin-saturated biotinylated MNPs 2554, biotinylated antisense oligonucleotides 2560, and analytes 2570 may be combined and/or mixed biological sample vial 2556.

In some examples, the biotinylated antisense oligonucleotides 2560 may be configured to bind and/or pair with analytes 2570, and the streptavidin-saturated biotinylated MNPs 2554, biotinylated antisense oligonucleotides 2560, and analytes 2570 may form clusters 2580. The clustering of MNPs may weaken the dynamic magnetic responses, and as a result, the harmonic amplitudes drop and/or reduce relative to well-dispersed MNPs. The difference in harmonic amplitude reduction may be used to quantitatively analyze the amount and concentration of analyte 2570 in a sample. An MPS-based bioassay, e.g., such as with MPS handheld device 102, may not require removing unbound target analytes 2570 and may be a wash-free, one-step test that is accessible by a layperson in nonclinical settings. MNP 2552 when bound to analytes 2570, may be an example of a bound MNP 704 (FIG. 7B) with an increased hydrodynamic size and with forming a cluster (e.g., with clustering with one or more other MNPs, e.g., forming one or more cluster 2580).

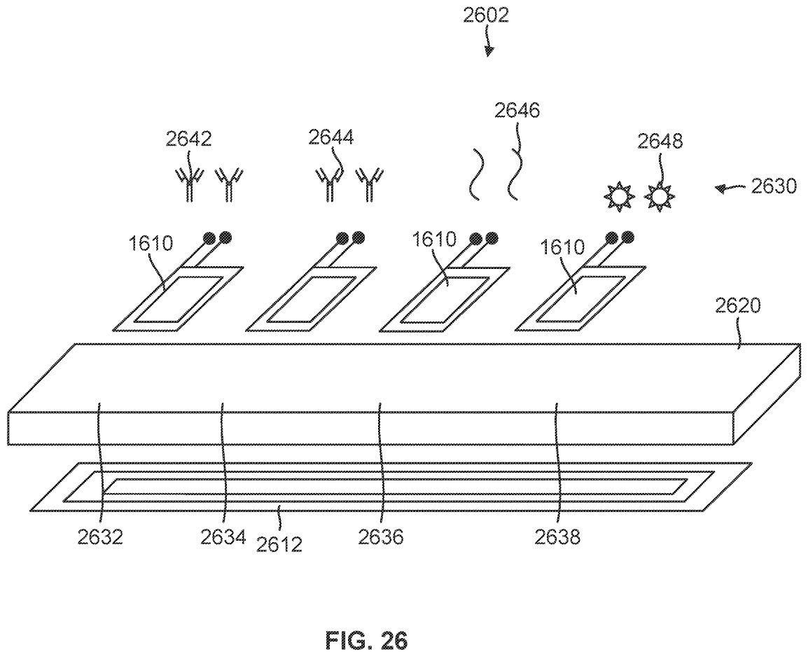

FIGS. 26-29 are diagrams illustrating examples of altered form MPS devices 2602 and 2702 and an example method 2900 of measuring a sample using MPS devices 2602 and 2702. In some examples, MPS devices 2602 and 2702 may use surface-bound antigens, antibodies, single strand DNA and/or RNA, and the like.

FIG. 26 is an illustration of an exploded-view of an example MPS device 2602, in accordance with one or more techniques of this disclosure. In the example shown, MPS device 2602 includes excitation coils 2612, sensing and/or pick-up coils 2616, testing strip 2620, and a chemical functionalization layer 2630. In some examples, MPS device 2602 may include a testing strip, and may contain one or more sensing regions, each region specific to a target analyte. In some examples, MPS device 2602 may be compatible with, or built into, a microfluidic channel and combined with system providing samples to test via microfluidic channels, e.g., a blood filtration system.

In the example shown, excitation coils 2612 are located on or adjacent to the bottom surface of testing strip 2620. Excitation coils 2612 may be substantially similar in function to excitation coils 1212 and/or 1214 illustrated and described above with respect to FIG. 12 and may include one or more windings similar to excitation coils 1212 and 1214. However, as shown in FIG. 26, excitation coils 2612 are substantially planar.

In the example shown in FIG. 26, chemical functionalization layer 2630 may include a plurality of sensing regions, e.g., sensing regions 2632-2638. Each sensing region may include a surface functionalization coating including a corresponding probe type, for example, a first antibody 2642, a second antibody 2644 different from first antibody 2642, single stranded DNA or RNA 2646, and antigen 2648. As such, each sensing region may detect a different analyte. Each of the plurality of probes may be configured for detecting, binding with, etc., a specific type of analyte. In some examples, chemical functionalization layer 2630 may be disposed on top of sensing coils 2616, each sensing coil 2616 corresponding to a sensing region 2632-2638.

In the example shown, sensing coils 2616 are located on the top surface of testing strip 2620, and may be substantially similar to pick-up coils 1216 illustrated and described above with respect to FIG. 12, aside from their substantially planar configuration. In the example shown, MPS device 2602 includes a sensing coil 2616 corresponding to each of the plurality of sensing regions, e.g., sensing regions 2632-2638, for example, to independently measure analytes being tested for corresponding to each of the plurality of regions.

In the example shown testing strip 2620 may provide structure for MPS device 2602 and a flow path for a biofluid to come in fluid communication with surface functionalization layer 2630. In some examples, testing strip 2620 may be and/or include a nitrocellulose membrane.

In the example shown, each probe type 2642-2648 of sensing region 2632-2638 may bind to a specific analyte type, and each bound analyte type may further bind to a surface functionalized MNP. For example, a fluid including a biologic sample including one or more analytes and one or more surface functionalized MNPs may be fluidically provided to testing strip 2620 and may come in fluid communication with surface functionalization layer 2630. The analytes corresponding to the specific probe types 2642-2648 may be bound and captured by the probes 2642-2648, and MNPs with surface functionalization corresponding to the captured analytes may be bound and captured to the captured analytes. In some examples, an alternating magnetic field may be generated by excitation coils 2612, and sensing coils 1610 may detect the magnetic response of the captured MNPs and generate a signal based on the magnetic response of the captured MNPs.

In some examples, MPS device 2602 may detect the magnetic moment of captured MNPs, e.g., sensing coil 2616 may generate a signal proportional to the magnetic moment of one or more captured MNPs within the alternating magnetic field. For example, MPS device 2602 may generate a signal including harmonics of the frequencies of the excitation coils, and the presence of the harmonics may be proportional to the magnetic moment of the plurality of captured MNPs. Because the motion of MNPs due to the alternating magnetic field is restricted via being bound to a surface of MPS device 2602, the magnetic response of MNPs of MPS device 2602 bound to the surface may not undergo Brownian motion and may not obey a Langevin function, e.g., in contrast to the operating principle of MPS handheld device 102 described above with respect to FIG. 13. In some examples, the MNPs of MPS device 2602 may be single-core or multi-core, and may be superparamagnetic.

In some examples, MPS device 2602 may communication with a computing device or devices, e.g., computing device 104, similar to MPS handheld device 102 as illustrated and described above with respect to FIG. 1, and may output a signal to computing device 104.

In some examples, MPS device 2602 may be a double-layered printed circuit board (PCB), or integrated into a double-layered PCB, or MPS device 2602 may be, or may be integrated into, a silicon substrate and/or a polymer substrate, e.g., via microfabrication. For example, coils 2612 and 1610 may be integrated into a double-layered PCB or micro-fabricated on a silicon substrate, and MPS device 2602 may be an adaptor card configured to be inserted into, and communicatively coupled to, a different MPS device, e.g., MPS device 3004 described and illustrated below, and/or a computing device.

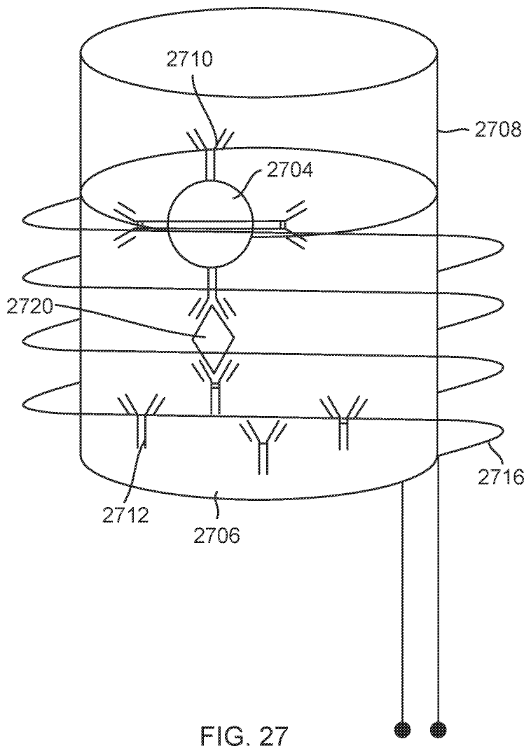

FIG. 27 is an illustration of an example MPS device 2702, in accordance with one or more techniques of this disclosure. In the example shown, MPS device 2702 includes coils 2716 and container 2708. In some examples, MPS device 2702 may be compatible with, or built into, a microfluidic channel and combined with a system providing samples to test via microfluidic channels, e.g., a blood filtration system.

In the example shown, coils 2716 are configured to coil around container 2708. Although only one set of coils is illustrated, coils 2716 may include primary, secondary, etc., excitation coils and one or more sets of sensing coils, e.g., pick-up coils. The excitation coils may be substantially similar to excitation coils 1212 and/or 1214 illustrated and described above with respect to FIG. 4A and may include one or more windings similar to excitation coils 1212 and 1214. The sensing coils may be substantially similar to pick-up coils 1216 illustrated and described above with respect to FIG. 4A.

In the example shown, container 2708 includes a chemical functionalization layer 2706. Chemical functionalization layer 2706 may be bound to one or more inner surfaces of container 2708, and may include a surface functionalization coating including a plurality of probe types, for example, antibodies, antigens, single stranded DNA or RNA, and the like. In the example shown, chemical functionalization layer 2706 is coated with antibodies 2712. Each of the plurality of probes, e.g., antibodies 2712, may be configured for detecting, binding with, etc., a specific type of analyte.

In the example shown, MPS device 2702 includes a plurality of MNPs, e.g., MNP 2704. MNP 2704 may be surface functionalized via a coating of a probe type, e.g., antibodies, antigens, single stranded DNA or RNA, and the like. In the examples shown, MNP 2704 is surface functionalized via a coating of antibodies 2710. In some examples, antibodies 2712 may correspond to antibodies 2710, that is, antibodies 2710 and 2712 may be configured for detecting, binding with, etc., the same specific type of analyte.

In operation, a biological sample may be added to MPS device 2702 including an analyte, e.g., an antibody, antigen, single stranded DNA or RNA, and the like. In the example shown, antigen 2720 has been added to container 2708. In some examples, the antibodies 2710 and 2712 may bind with antigen 2720, thereby binding MNP 2704 to chemical functionalization layer 2706.

In some examples, MPS device 2702 may communication with a computing device or devices, e.g., computing device 104, similar to MPS handheld device 102 as illustrated and described above with respect to FIG. 1. In some examples, the operating principles of MPS device 2702 may be similar to that illustrated described above with respect to FIGS. 12-13.

Accordingly, although example systems and techniques have been shown and described, it is to be understood that all the terms used herein are descriptive rather than limiting, and that many changes, modifications, and substitutions may be made by one having ordinary skill in the art without departing from the spirit and scope of the invention. The following examples are examples of systems, devices, and methods described herein.

Example 1: A bioassay system comprising: at least one conductive excitation coil, the at least one conductive excitation coil configured to generate an alternating magnetic field including a first frequency and a second frequency; a sample mount configured to position a sample within the at least one conductive excitation coil; a separator configured to separate a bound magnetic nanoparticle (MNP) from an unbound MNP, wherein the bound MNP comprises a surface functionalized MNP bound to an analyte, wherein the unbound MNP comprises a surface functionalized MNP not bound to an analyte, wherein the sample comprises the bound MNP and does not comprise the unbound MNP; and at least one sensing conductive coil configured to sense a magnetic response of the sample positioned within the sample mount to the alternating magnetic field.

Example 2: The bioassay system of claim 1, wherein the magnetic response of the sample comprises a Néel relaxation response.

Example 3: The bioassay system of example 2, wherein the Néel relaxation response of the sample comprises a higher order harmonic amplitude response that is greater than a higher order harmonic amplitude response of a Brownian relaxation response of the sample.

Example 4: The bioassay system of any one of examples 1-3, wherein the separator is configured to separate a plurality of unbound MNPs from a plurality of bound MNPs, wherein the sample comprises at least a portion of the bound MNPs and does not comprise at least a portion of the unbound MNPs.

Example 5: The bioassay system of any one of examples 1-4, wherein the surface functionalization of the bound MNP and the unbound MNP includes a coating comprising a probe configured to capture an analyte, wherein capturing the analyte changes a hydrodynamic size and the magnetic response of the bound MNP relative to the unbound MNP.

Example 6: The bioassay system of example 5, wherein the probe comprises at least one of an antigen, an antibody, a single stranded deoxyribonucleic acid (DNA), a single stranded ribonucleic acid (RNA), an antisense nucleotide, or a peptide.

Example 7: The bioassay system of example 5 or example 6, wherein the analyte comprises at least one of an antigen, an antibody, a single stranded DNA, and a single stranded RNA, a heavy metal ion, or a protease.

Example 8: The bioassay system of example 7, wherein the analyte comprises at least one of human coronavirus 229E, human coronavirus OC43, SARS-COV (2003), HCOV NL63 (2004), HKU1 (2005), MERS-COV (2102), or SARS-COV-2.

Example 9: The bioassay system of any one of examples 5-8, wherein the sample comprises a plurality of bound MNPs bound in a cluster via capture of one or more analytes.

Example 10: The bioassay system of any one of examples 1-9, wherein the magnetic response of the sample comprises a plurality of higher order harmonics, wherein a ratio of an amplitude of a first higher order harmonic to a second higher order harmonic is proportional to the binding of analytes to MNPs.

Example 11: The bioassay system of any one of examples 1-10, wherein the bound MNP comprises a nonmagnetic microstructure configured to increase a surface area of the bound MNP.

Example 12: The bioassay system of example 11, wherein the nonmagnetic microstructure comprises at least one of a microbead, a microrod, or a non-spherical microstructure.

Example 13: The bioassay system of example 11 or example 12, wherein the nonmagnetic microstructure comprises at least one of silicon, gold, or a polymer.

Example 14: The bioassay system of any one of examples 1-13, wherein the separator is configured to separate the bound MNP from the unbound MNP by at least one of a filter, an acoustic wave, a gravitation force, a centrifugal force, or a magnetic force.

Example 15: A method comprising: positioning a sample within at least one conductive excitation coil, wherein the at least one conductive excitation coil is configured to generate an alternating magnetic field including a first frequency and a second frequency; separating a bound magnetic nanoparticle (MNP) from an unbound MNP, wherein the bound MNP comprises a surface functionalized MNP bound to an analyte, wherein the unbound MNP comprises a surface functionalized MNP not bound to an analyte, wherein the sample comprises the bound MNP and does not comprise the unbound MNP; and sensing, by at least one sensing conductive coil, a magnetic response of the sample to the alternating magnetic field.

Example 16: The method of example 15, wherein the magnetic response of the sample comprises a Néel relaxation response.

Example 17: The method of example 16, wherein the Neel relaxation response of the sample comprises a higher order harmonic amplitude response that is greater than a higher order harmonic amplitude response of a Brownian relaxation response of the sample.

Example 18: The method of any one of examples 15-17, wherein separating the bound magnetic nanoparticle (MNP) from the unbound MNP comprises separating a plurality of unbound MNPs from a plurality of bound MNPs, wherein the sample comprises at least a portion of the bound MNPs and does not comprise at least a portion of the unbound MNPs.

Example 19: The method of any one of examples 15-18, wherein the surface functionalization of the bound MNP and the unbound MNP includes a coating comprising a probe configured to capture an analyte, wherein capturing the analyte changes a hydrodynamic size and the magnetic response of the bound MNP relative to the unbound MNP.

Example 20: The method of example 19, wherein the probe comprises at least one of an antigen, an antibody, a single stranded deoxyribonucleic acid (DNA), a single stranded ribonucleic acid (RNA), an antisense nucleotide, or a peptide.

Example 21: The method of example 19 or example 20, wherein the analyte comprises at least one of an antigen, an antibody, a single stranded DNA, and a single stranded RNA, a heavy metal ion, or a protease.

Example 22: The method of example 21, wherein the analyte comprises at least one of human coronavirus 229E, human coronavirus OC43, SARS-COV (2003), HCOV NL63 (2004), HKU1 (2005), MERS-COV (2102), or SARS-COV-2.

Example 23: The method of any one of examples 19-22, wherein the sample comprises a plurality of bound MNPs bound in a cluster via capture of one or more analytes.

Example 24: The method of example 23, wherein the magnetic response of the sample comprises a plurality of higher order harmonics, wherein a ratio of an amplitude of a first higher order harmonic to a second higher order harmonic is proportional to the binding of analytes to MNPs.

Example 25: The method of any one of examples 15-24, wherein the bound MNP comprises a nonmagnetic microstructure configured to increase a surface area of the bound MNP.

Example 26: The method of example 25, wherein the nonmagnetic microstructure comprises at least one of a microbead, a microrod, or a non-spherical microstructure.

Example 27: The method of example 25 or example 26, wherein the nonmagnetic microstructure comprises at least one of silicon, gold, or a polymer.

Example 28: The method of any one of examples 15-27, wherein separating the bound MNP from the unbound MNP comprises at least one of filtering the bound MNP from the unbound MNP, separating the bound MNP from the unbound MNP via an acoustic wave, separating the bound MNP from the unbound MNP via a gravitation force, separating the bound MNP from the unbound MNP via a centrifugal force, or separating the bound MNP from the unbound MNP via a magnetic force.

Example 29: A bioassay system comprising: at least one conductive excitation coil, the at least one conductive excitation coil configured to generate an alternating magnetic field including a first frequency and a second frequency; a sample mount configured to position a sample within the at least one conductive excitation coil, wherein the sample comprises a bound magnetic nanoparticle (MNP) that has been separated from an unbound MNP, wherein the sample does not comprise the unbound MNP, wherein the bound MNP comprises a surface functionalized MNP bound to an analyte, wherein the unbound MNP comprises a surface functionalized MNP not bound to an analyte; at least one sensing conductive coil configured to sense a magnetic response of the sample positioned within the sample mount to the alternating magnetic field.

Example 30: The bioassay system of example 29, wherein the sample comprises a plurality of bound MNPs, wherein a ratio of a first amount of bound MNPs to a first amount of unbound MNPs comprising the sample is greater than a ratio of a second amount of bound MNPs to a second amount of unbound MNPs before the separating bound MNPs from unbound MNPs.

Example 31: The bioassay system of example 29 or example 30, wherein the magnetic response of the sample comprises a Néel relaxation response.

Example 32: The bioassay system of example 31, wherein the Néel relaxation response of the sample comprises a higher order harmonic amplitude response that is greater than a higher order harmonic amplitude response of a Brownian relaxation response of the sample.

Example 33: The bioassay system of any one of examples 29-32, wherein the bound MNP comprises a surface functionalized MNP and the unbound MNP comprises a surface functionalized MNP, wherein the surface functionalization of the bound MNP and the unbound MNP includes a coating comprising a probe configured to capture an analyte, wherein capturing the analyte changes a hydrodynamic size and the magnetic response of the bound MNP relative to the unbound MNP.

Example 34: The bioassay system of example 33, wherein the probe comprises at least one of an antigen, an antibody, a single stranded deoxyribonucleic acid (DNA), a single stranded ribonucleic acid (RNA), an antisense nucleotide, or a peptide.

Example 35: The bioassay system of example 33 or example 34, wherein the analyte comprises at least one of an antigen, an antibody, a single stranded DNA, and a single stranded RNA, a heavy metal ion, or a protease.

Example 36: The bioassay system of example 35, wherein the analyte comprises at least one of human coronavirus 229E, human coronavirus OC43, SARS-COV (2003), HCOV NL63 (2004), HKU1 (2005), MERS-COV (2102), or SARS-COV-2.

Example 37: The bioassay system of any one of examples 33-36, wherein the sample comprises a plurality of bound MNPs bound in a cluster via capture of one or more analytes.

Example 38: The bioassay system of any one of examples 29-37, wherein the magnetic response of the sample comprises a plurality of higher order harmonics, wherein a ratio of an amplitude of a first higher order harmonic to a second higher order harmonic is proportional to the binding of analytes to MNPs.

Example 39: The bioassay system of any one of examples 29-38, wherein the bound MNP comprises a nonmagnetic microstructure configured to increase a surface area of the bound MNP.

Example 40: The bioassay system of example 39, wherein the nonmagnetic microstructure comprises at least one of a microbead, a microrod, or a non-spherical microstructure.

Example 41: The bioassay system of example 39 or example 40, wherein the nonmagnetic microstructure comprises at least one of silicon, gold, or a polymer.

Example 42: The bioassay system of any one of examples 29-41, wherein the bound MNP and the unbound MNP are separated by at least one of a filter, an acoustic wave, a gravitation force, a centrifugal force, or a magnetic force.

Example 43: A method comprising: separating a bound magnetic nanoparticle (MNP) from an unbound MNP, wherein the bound MNP comprises an MNP bound to an analyte, wherein the bound MNP comprises a surface functionalization including a probe configured to bind to the analyte, wherein the bound MNP has a hydrodynamic size that is larger than the unbound MNP.

Example 44: The method of example 43, wherein separating the bound MNP from the unbound MNP comprises filtering the bound MNP from the unbound MNP by size.

Example 45: The method of example 44, wherein a filter for the filtering has a pore size that is less than the hydrodynamic size of the bound MNP and greater than a hydrodynamic size of the unbound MNP.

Example 46: The method of any one of examples 43-45, wherein separating the bound MNP from the unbound MNP comprises: causing the bound MNP and the unbound MNP to flow in a sample fluid; joining the flow of the sample fluid with a flow of a sheath fluid, wherein the sheath fluid does not comprise bound MNPs or unbound MNPs; causing a surface acoustic wave to form in the joined flow of sample fluid and the sheath fluid, wherein the surface acoustic wave is configured to cause particles having a hydrodynamic size greater than a threshold hydrodynamic size to move within a fluid in a non-flow direction, wherein the bound MNP has a hydrodynamic size that is greater than or equal to the threshold hydrodynamic size, wherein the unbound MNP has a hydrodynamic size that is less than the threshold hydrodynamic size; and separating the flow of the sheath fluid from the flow of the sample fluid downstream from the surface acoustic wave.

Example 47: The method of example 46, wherein the bound MNP within the flow of the sample fluid moves to the flow of the sheath fluid upstream from separating the flow of the sheath fluid from the flow of the sample fluid.

Example 48: The method of any one of examples 43-47, wherein separating the bound MNP from the unbound MNP comprises: causing the bound MNP and the unbound MNP to flow in a sample fluid within a channel comprising a first outlet and a second outlet, wherein the first outlet is positioned proximate to a first side of the cross-sectional area of the channel, wherein the second outlet is positioned opposite the first outlet from the first side and in the direction of a gravitational force, wherein the direction of the gravitational force is substantially perpendicular to the flow of the sample fluid.

Example 49: The method of example 48, wherein the bound MNP within the flow of the sample fluid moves in the direction of the gravitational force more than the unbound MNP.

Example 50: The method of any one of examples 43-49, wherein separating the bound MNP from the unbound MNP comprises: centrifuging the bound MNP and the unbound MNP within a fluid to cause the bound MNP to sediment to a bottom portion of the fluid while the unbound MNP remains in a supernatant portion of the fluid; and removing the supernatant portion from the sediment portion.

Example 51: The method of any one of examples 43-50, wherein separating the bound MNP from the unbound MNP comprises: causing the bound MNP and the unbound MNP to flow in a sample fluid within a channel comprising a first outlet and a second outlet, wherein the first outlet is positioned proximate to a first side of the cross-sectional area of the channel, wherein the second outlet is positioned opposite the first outlet from the first side; and applying a gradient magnetic field to a portion of the length of the channel upstream from the first and second outlets, the gradient magnetic field configured to cause a magnetic force on the bound MNP and the unbound MNP in a direction substantially perpendicular to the direction of the flow of the fluid.

Example 52: The method of any one of examples 48-52, wherein a fluid resistance of the fluid is proportional to hydrodynamic size causing the unbound MNP to move in the direction of the magnetic force by a greater amount than the bound MNP.

Example 53: A separation apparatus comprising: a container configured to house a sample comprising a bound magnetic nanoparticle (MNP) and an unbound MNP, wherein the bound MNP comprises an MNP bound to an analyte, wherein the bound MNP comprises a surface functionalization including a probe configured to bind to the analyte, wherein the bound MNP has a hydrodynamic size that is larger than the unbound MNP; and a separator configured to separate, based on hydrodynamic size, the bound MNP from the unbound MNP.

Example 54: The separation apparatus of example 53, wherein the separator comprises a filter.

Example 55: The separation apparatus of example 54, wherein a filter for the filtering has a pore size that is less than the hydrodynamic size of the bound MNP and greater than a hydrodynamic size of the unbound MNP

Example 56: The separation apparatus of any one of examples 53-55, wherein the sample comprises the bound MNP and the unbound MNP within a sample fluid, wherein the separator comprises: a channel in fluid communication with the container and in fluid communication with a flow of a sheath fluid, wherein the sheath fluid does not comprise bound or unbound MNPs; an interdigital transducer configured to cause a surface acoustic wave within a flow of the sample fluid and the sheath fluid.

Example 57: The separation apparatus of example 56, wherein the surface acoustic wave is configured to cause the bound MNP within the flow of the sample fluid move to the flow of the sheath fluid.

Example 58: The separation apparatus of any one of examples 53-57, wherein the sample comprises the bound MNP and the unbound MNP within a sample fluid, wherein the separator comprises: a channel in fluid communication with the container, the channel comprising a first outlet and a second outlet, wherein the first outlet is positioned proximate to a first side of the cross-sectional area of the channel, wherein the second outlet is positioned opposite the first outlet from the first side and in the direction of a gravitational force, wherein the direction of the gravitational force is substantially perpendicular to a flow of the sample fluid.

Example 59: The separation apparatus of example 58, wherein the bound MNP within the flow of the sample fluid moves in the direction of the gravitational force more than the unbound MNP.

Example 60: The separation apparatus of any one of examples 53-59, wherein the sample comprises the bound MNP and the unbound MNP within a sample fluid, wherein the separator comprises: a centrifuge configured to cause the bound MNP to sediment to a bottom portion of the sample fluid while the bound MNP remains in a supernatant portion of the sample fluid.

Example 61: The separation apparatus of any one of examples 53-60, wherein the sample comprises the bound MNP and the unbound MNP within a sample fluid, wherein the separator comprises: a channel in fluid communication with the container, the channel comprising a first outlet and a second outlet, wherein the first outlet is positioned proximate to a first side of the cross-sectional area of the channel, wherein the second outlet is positioned opposite the first outlet from the first side; and a magnet configured to apply a gradient magnetic field to a portion of the length of the channel upstream from the first and second outlets, the gradient magnetic field configured to cause a magnetic force on the bound MNP and the unbound MNP in a direction substantially perpendicular to the direction of a flow of the sample fluid, wherein a fluid resistance of the sample fluid is proportional to hydrodynamic size causing the unbound MNP to move in the direction of the magnetic force by a greater amount than the bound MNP.

Example 62: A magnetic nanoparticle (MNP) test material comprising: a surface functionalized MNP comprising a surface functionalization including a probe configured to bind to an analyte; and a nonmagnetic microstructure configured to bind to the analyte.

Example 63: The MNP test material of example 62, wherein the nonmagnetic microstructure comprises at least one of a microbead, a microrod, or a non-spherical microstructure.

Example 64: The MNP test material of example 63, wherein the nonmagnetic microstructure comprises the microrod, wherein the microrod comprises a length that is greater than twice its width and height.

Example 65: The MNP test material of example 63, wherein the nonmagnetic microstructure comprises the non-spherical microstructure, wherein the non-spherical microstructure comprises an irregular shape.

Example 66: The MNP test material of example 63 or example 65, wherein the nonmagnetic microstructure comprises the non-spherical microstructure, wherein the non-spherical microstructure comprises a polygonal shape.

Example 67: The MNP test material of any one of examples 62-66, wherein the MNP test material is configured to form a cluster comprising a surface functionalized MNPs bound with the nonmagnetic structure via capture of the analyte upon combination with a biological sample including the analyte.

Example 68: The MNP test material of example 67, wherein the nonmagnetic microstructure is configured to increase a hydrodynamic size of the surface functionalized MNP.

The techniques described in this disclosure may be implemented, at least in part, in hardware, software, firmware or any combination thereof. For example, various aspects of the described techniques may be implemented within one or more processors, including one or more microprocessors, DSPs, application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

What is claimed is:

1. A method comprising:
separating a bound magnetic nanoparticle (MNP) from an unbound MNP, wherein the bound MNP comprises an MNP bound to an analyte,
wherein the bound MNP comprises a surface functionalization including a probe configured to bind to the analyte.

2. The method of claim 1, wherein the bound MNP has a hydrodynamic size that is larger than the unbound MNP, wherein separating the bound MNP from the unbound MNP comprises filtering the bound MNP from the unbound MNP by size.

3. The method of claim 2, wherein a filter for the filtering has a pore size that is less than the hydrodynamic size of the bound MNP and greater than a hydrodynamic size of the unbound MNP.

4. The method of claim 1, wherein separating the bound MNP from the unbound MNP comprises:
causing the bound MNP and the unbound MNP to flow in a sample fluid within a channel comprising a first outlet and a second outlet,
wherein the first outlet is positioned proximate to a first side of the cross-sectional area of the channel, wherein the second outlet is positioned opposite the first outlet from the first side and in the direction of a gravitational force, wherein the direction of the gravitational force is substantially perpendicular to the flow of the sample fluid.

5. The method of claim 4, wherein the bound MNP within the flow of the sample fluid moves in the direction of the gravitational force more than the unbound MNP.

6. The method of claim 1, wherein separating the bound MNP from the unbound MNP comprises:
causing the bound MNP and the unbound MNP to flow in a sample fluid within a channel comprising a first outlet and a second outlet,
wherein the first outlet is positioned proximate to a first side of the cross-sectional area of the channel, wherein the second outlet is positioned opposite the first outlet from the first side; and
applying a gradient magnetic field to a portion of the length of the channel upstream from the first and second outlets, the gradient magnetic field configured to cause a magnetic force on the bound MNP and the unbound MNP in a direction substantially perpendicular to the direction of the flow of the fluid.

7. The method of claim 4, wherein a fluid resistance of the fluid is proportional to hydrodynamic size causing the unbound MNP to move in the direction of the magnetic force by a greater amount than the bound MNP.

8. A separation apparatus comprising:
a container configured to house a sample comprising a bound magnetic nanoparticle (MNP) and an unbound MNP, wherein the bound MNP comprises an MNP bound to an analyte, wherein the bound MNP comprises a surface functionalization including a probe configured to bind to the analyte, wherein the bound MNP has a hydrodynamic size that is larger than the unbound MNP; and
a separator configured to separate, based on hydrodynamic size, the bound MNP from the unbound MNP.

9. The separation apparatus of claim 8, wherein the separator comprises a filter.

10. The separation apparatus of claim 9, wherein a filter for the filtering has a pore size that is less than the hydrodynamic size of the bound MNP and greater than a hydrodynamic size of the unbound MNP.

11. The separation apparatus of claim 8, wherein the sample comprises the bound MNP and the unbound MNP within a sample fluid, wherein the separator comprises:
a channel in fluid communication with the container, the channel comprising a first outlet and a second outlet, wherein the first outlet is positioned proximate to a first side of the cross-sectional area of the channel, wherein the second outlet is positioned opposite the first outlet from the first side and in the direction of a gravitational force, wherein the direction of the gravitational force is substantially perpendicular to a flow of the sample fluid.

12. The separation apparatus of claim 11, wherein the bound MNP within the flow of the sample fluid moves in the direction of the gravitational force more than the unbound MNP.

13. The separation apparatus of claim 8, wherein the sample comprises the bound MNP and the unbound MNP within a sample fluid, wherein the separator comprises: a centrifuge configured to cause the bound MNP to sediment to a bottom portion of the sample fluid while the bound MNP remains in a supernatant portion of the sample fluid.

14. The separation apparatus of claim 8, wherein the sample comprises the bound MNP and the unbound MNP within a sample fluid, wherein the separator comprises:
a channel in fluid communication with the container, the channel comprising a first outlet and a second outlet, wherein the first outlet is positioned proximate to a first side of the cross-sectional area of the channel, wherein the second outlet is positioned opposite the first outlet from the first side; and
a magnet configured to apply a gradient magnetic field to a portion of the length of the channel upstream from the first and second outlets, the gradient magnetic field configured to cause a magnetic force on the bound MNP and the unbound MNP in a direction substantially perpendicular to the direction of a flow of the sample fluid, wherein a fluid resistance of the sample fluid is proportional to hydrodynamic size causing the unbound MNP to move in the direction of the magnetic force by a greater amount than the bound MNP.

15. A magnetic nanoparticle (MNP) test material comprising:
a surface functionalized MNP comprising a surface functionalization including a probe configured to bind to an analyte; and
a nonmagnetic microstructure configured to bind to the analyte.

16. The MNP test material of claim 15, wherein the nonmagnetic microstructure comprises at least one of a microbead, a microrod, or a non-spherical microstructure.

17. The MNP test material of claim 16, wherein the nonmagnetic microstructure comprises the microrod, wherein the microrod comprises a length that is greater than twice its width and height.

18. The MNP test material of claim 16, wherein the nonmagnetic microstructure comprises the non-spherical microstructure, wherein the non-spherical microstructure comprises an irregular shape.

19. The MNP test material of claim 15, wherein the MNP test material is configured to form a cluster comprising a surface functionalized MNPs bound with the nonmagnetic structure via capture of the analyte upon combination with a biological sample including the analyte.

20. The MNP test material of claim 19, wherein the nonmagnetic microstructure is configured to increase a hydrodynamic size of the surface functionalized MNP.
